(19) United States
(12) Patent Application Publication
(10) Pub. No.: US 2008/0051451 A1
(43) Pub. Date: Feb. 28, 2008

(54) Autoimmune conditions and NADPH oxidase defects

(76) Inventors: Marcel Linschoten, Haninge (SE); Anders Bergman, Lindome (SE); Tolmachev Andrey Alekseevich, Kiev (UA); Peter Olofsson, Vastra Frolunda (SE)

Correspondence Address: FISH & RICHARDSON PC, P.O. BOX 1022, MINNEAPOLIS, MN, 55440-1022, US

(21) Appl. No.: 11/724,354

(22) Filed: Mar. 15, 2007

Related U.S. Application Data
(60) Provisional application No. 60/782,381, filed on Mar. 15, 2006.

(51) Int. Cl. A61K 31/352; A61K 31/045; A61P 37/00; A61K 31/22

(52) U.S. Cl. 514/456; 514/549; 514/739

(57) ABSTRACT

The invention relates to methods and materials involved in treating autoimmune conditions. In particular, the invention relates to methods and compounds (and compositions including the compounds) involved in treating, preventing, or delaying the onset of autoimmune conditions. The compounds include one or more isoprenoid units. The compounds can enhance NADPH oxidase activity.

### CROSS REFERENCE TO RELATED APPLICATIONS

This application claims the benefit of U.S. Provisional Application No.: 60/782,381, filed on Mar. 15, 2006, which is incorporated herein by reference in its entirety.

### BACKGROUND

1. Technical Field

The present disclosure relates to methods and materials involved in treating, preventing, and ameliorating one or more conditions associated with autoimmune conditions. In particular, the present disclosure relates to methods and materials involved in treating, preventing, or delaying the onset of arthritis and multiple sclerosis.

2. Background Information

Autoimmune conditions are conditions where a mammal's immune system reacts against its own tissues. Such conditions include, without limitation, arthritis (e.g., rheumatoid arthritis (RA)), multiple sclerosis (MS), inflammatory bowel disease, Crohn disease, lupus, autoimmune uveitis, type I diabetes, bronchial asthma, septic arthritis induced with staphylococci or streptococci, and cardiovascular disease involving vasculitis.

RA is a chronic inflammatory disease that can be found in about 1-2% of the population. RA primarily affects peripheral joints where inflammatory synovitis leads to cartilage destruction, bone erosion, and ultimately to joint deformity and loss of joint function. RA is a complex disease in that both environmental factors as well as multiple chromosomal regions are involved in susceptibility to RA. Inducers of arthritis in animal models include adjuvants, collagen (e.g., collagen type II) (collagen induced arthritis (CIA)), hexadecane (hexadecane induced arthritis (HIA)), oil (e.g., Freund's incomplete adjuvant), squalene (squalene induced arthritis (SIA), and pristane (pristane induced arthritis (PIA)). Chromosomal regions known to be associated with development of RA include the major histocompatibility complex region. In addition, different genomic regions are known to control different phases of the disease such as onset, severity during the acute onset phase, and the severity of the destruction in the chronic relapsing phase.

MS is a chronic inflammatory disease of the central nervous system. The characteristic pathological feature is demyelination of the myelin sheath of neurons in the central nervous system, resulting in multiple and varied neurologic symptoms and signs, usually with repeated relapse and remission. MS affects more than 2 million people worldwide. Studies have implicated a cell-mediated immune response, involving T cells recognizing epitopes of myelin basic protein (MBP), in the pathogenesis of MS. Experimental autoimmune encephalomyelitis (EAE) is an autoimmune inflammatory and demyelinating disease model that shares many clinical and histological features with MS. EAE can be induced in susceptible strains of mice by immunization with self antigens derived from myelin.

The Ncf1 gene, encoding the p47phox subunit of the NADPH oxidase complex, has been shown to be associated with the development of arthritis in the DA rat. (Olofsson et al. (2003) Nature Genetics 33:25-32; WO 03/095667), and the development of arthritis and encephalomyelitis in mice (Hultqvist et al. (2004) Proc Natl Acad Sci USA. 101:12646-51).

### SUMMARY

Provided herein are methods and materials related to treating autoimmune conditions such as arthritis (e.g., RA), multiple sclerosis, lupus, autoimmune uveitis, type I diabetes, bronchial asthma, septic arthritis induced with staphylococci or streptococci, and cardiovascular disease involving vasculitis. For example, this disclosure provides methods and materials involved in treating, preventing, ameliorating one or more symptoms associated with, and/or delaying the onset of autoimmune conditions.

The inventions described herein are based on the discovery that arthritis can be associated with or caused by a reduced level of NADPH oxidase activity. For example, development of severe arthritis symptoms in an arthritis animal model can be, at least partially, dependent upon the presence of low NADPH oxidase activity. In addition, the inventions are based on the discovery that mammals prone to develop arthritis can be protected by providing those mammals with normal or increased levels of NADPH oxidase activity.

The inventions are also based on the discovery that compounds that include one or more isoprenoid units (e.g., one or more unsaturated isoprenoid units and/or one or more saturated isoprenoid units) can be used as NADPH activators. NADPH activators can be useful for treating, lessening the severity of the symptoms of, preventing, or delaying the onset of symptoms of an autoimmune condition in an animal such as a mammal. Typically, the compounds include at least one unsaturated isoprenoid unit or saturated isoprenoid unit that is derivatized with a functional moiety. A variety of compound classes useful as NADPH activators are described in more detail below (e.g., in compound classes 1-46).

The invention features compounds; compositions (e.g., pharmaceutical compositions) containing the compounds; and methods for treating a mammal having an autoimmune condition (e.g., arthritis or multiple sclerosis) or ameliorating one or more symptoms of the autoimmune condition in such a mammal. In some embodiments, the compounds described herein for treating a mammal can be NADPH oxidase activators. In some embodiments, the compounds can include up to 10, e.g., 1, 2, 3, 4, 5, 6, 7, 8, 9, or 10, e.g., 2 or 3, isoprenoid units (e.g., unsaturated isoprenoid units and/or one or more saturated isoprenoid units) conjugated to one another, in which the isoprenoid units can be arranged in any sequence or directionality (e.g., head to tail, head to head, tail to tail) relative to one another. One or more of the isoprenoid units (e.g., one or more unsaturated isoprenoid units and/or one or more saturated isoprenoid units) can be derivatized with one or more functional moieties. The compounds can include derivatized isoprenoid units (e.g., derivatized unsaturated isoprenoid units and/or derivatized saturated isoprenoid units) arranged in any sequence or directionality relative to one another and in any sequence or directionality relative to underivatized isoprenoid units (e.g., underivatized unsaturated isoprenoid and/or underivatized saturated isoprenoid units). Particular compound classes are described in more detail below (e.g., in compound classes 1-46).

The compounds may be administered intra-dermally, intra-peritoneally, orally, or intra-nasally.

In another embodiment, the invention features the use of one or more of the compounds described herein in the manufacture of a medicament to treat, prevent, delay the onset of one or more symptoms, or ameliorate one or more symptoms of an autoimmune condition (e.g., arthritis or multiple sclerosis), wherein the compound enhances NADPH oxidase activity in a mammal. The compounds can have any of the structural formulae delineated herein including, e.g., the structural formulae associated with compound classes 1-46.

Another embodiment of the invention features a method of formulating a medicament for the treatment of an autoimmune condition, the method including: (a) contacting a sample comprising cells or a cellular fraction having NADPH activity with a test composition, (b) determining the level of NADPH oxidase activity in the sample, (c) determining whether or not the level is greater than a control level of NADPH oxidase activity, wherein the control level is the amount of NADPH oxidase activity in a control sample lacking the test composition, (d) identifying the test composition as a composition useful for treatment of the autoimmune condition when the level of NADPH oxidase activity is greater than the control level, and (e) formulating a medicament from the composition for the treatment of the autoimmune condition. The autoimmune condition can be arthritis or multiple sclerosis. Test compounds can have any of the structural formulae delineated herein including, e.g., the structural formulae delineated in compound classes 1-46.

In one aspect, this invention features a method for treating an autoimmune condition in a mammal (e.g. a mammal in need thereof), the method includes administering to said mammal a compound (or a pharmaceutically acceptable salt of the compound), which includes one or more isoprenoid units.

In one aspect, this invention features a method for treating an autoimmune condition in a mammal (e.g. a mammal in need thereof), the method includes administering to said mammal a compound, or a pharmaceutically acceptable salt thereof, that enhances NADPH oxidase activity, wherein said compound includes one or more isoprenoid units.

In one aspect, this invention features a method for treating a mammal having an autoimmune condition, the method includes administering to the mammal (e.g., a mammal in need thereof) a compound, or a pharmaceutically acceptable salt thereof, that enhances NADPH oxidase activity, wherein said compound includes one or more isoprenoid units. The autoimmune condition can be, e.g., arthritis or multiple sclerosis.

In another aspect, this invention features the compounds delineated herein (e.g., genera, subgenera, or specific compounds set forth in compound classes 1-46).

In a further aspect, this invention features a pharmaceutical composition, which includes one or more of the compounds delineated herein (e.g., genera, subgenera, or specific compounds set forth in compound classes 1-46) or a salt (e.g., a pharmaceutically acceptable salt) or a prodrug thereof and a pharmaceutically acceptable adjuvant, carrier or diluent. In some embodiments, the composition can include an effective amount of the compound or the salt thereof. In some embodiments, the composition can further include an additional therapeutic agent.

Embodiments can include one or more of the following features.

The compound can include one or more unsaturated isoprenoid units. The compound can include one or more saturated isoprenoid units. In embodiments, at least one of said unsaturated isoprenoid units or saturated isoprenoid units can be derivatized with a functional moiety.

The compound can have the following general formula:

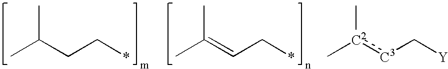

in which:

m+n=an integer from 0-9 (e.g., 0-5, 1-5, 2-5, 2-4, 2 or 3; e.g., one of m and n can be 0, and the other can be an integer from 1-9, e.g., 1-5, 1-4, 2-5, 2-4, 2 or 3; e.g., m+n=0);

[symbol] represents a single or double bond between C² and C³; and

Y can be a moiety, e.g., a functional moiety, that is present in the structural formulae associated with any of compound classes 1-46 (e.g., compound classes 1, 2, 13, 45, or 46). In embodiments, when variables R′, R″, and R′″ are present, each of these variables can be independently of one another, H, C₁-C₂₀ (e.g., C₁-C₁₂, C₁-C₁₀, C₁-C₆, or C₁-C₃) alkyl, C₂-C₂₀ (e.g., C₂-C₁₂, C₂-C₁₀, C₂-C₆, or C₂-C₄) alkenyl or alkynyl, C₃-C₂₀ (e.g., C₃-C₁₀, C₃-C₈, C₃-C₆) cycloalkyl, heterocyclyl including 3-20 (e.g., 3-10, 3-8, 3-6 atoms) atoms, C₆-C₁₈ (e.g., C₆-C₁₄, C₆-C₁₀, C₆) aryl, or heteroaryl including 3-20 (e.g., 3-10, 3-8, 3-6 atoms) atoms, each of which being optionally substituted as described herein.

In embodiments, Y can be —OR′ or —O—C(═O)R′, in which R′ can be H, alkyl, alkenyl, alkynyl, cycloalkyl, heterocyclyl, aryl, or heteroaryl, each of which is optionally substituted (e.g., Y can be hydrogen; optionally substituted alkyl, optionally substituted aryl; or heteroaryl).

In embodiments, Y can be OR′. R′ can be hydrogen.

In embodiments, Y can be —O—C(═O)R′. R′ can be C₁-C₆ alkyl (e.g., CH₃).

In embodiments, one of m and n can be 0. The other of m and n can be an integer from 1-9, e.g., 1-5, 1-4, 2-5, 2-4, 2 or 3 (e.g., 2-4). For example, n can be 0, and m can be an integer from 1-9, e.g., 1-5, 1-4, 2-5, 2-4, 2 or 3. As another example, m can be 0, and n can be an integer from 1-9, e.g., 1-5, 1-4, 2-5, 2-4, 2 or 3.

In embodiments, m+n=3. One of m and n can be 0 (e.g., n can be 0, and m can be 3 or vice versa).

In embodiments, m+n=2. One of m and n can be 0 (e.g., n can be 0, and m can be 2 or vice versa).

In embodiments, [symbol] can represent a double bond between C² and C³; or a single bond between C² and C³.

The compound can be selected from the group consisting of:

Phytol (3,7,11,15-Tetramethyl-2-hexadecen-1-ol);

Farnesol (3,7,11-Trimethyl-2,6, 10-dodecatrien-1-ol);

Geranylgeraniol (3,7,11,15-Tetramethyl-hexada-2,6,10,14-tetraen-1-ol);

Farnesyl acetate (Acetic acid 3,7,11-trimethyl-dodeca-2,6,10-trienyl ester);

Phytyl acetate (Acetic acid 3,7,11,15-tetra-metyl-hexadec-2-enyl-ester);

Di-hydro-phytol (3,7,11,15-Tetramethyl-hexadecan-1-ol); and

Di-hydro-phytyl acetate (Acetic acid 3,7,11,15-tetramethyl-hexadecyl ester);

In certain embodiments, the compound can be Phytol (3,7,11,15-Tetramethyl-2-hexadecen-1-ol).

The compound can have the general formula:

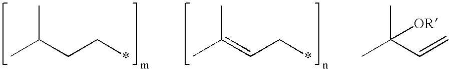

m+n=an integer from 0-9 (e.g., 0-5, 1-5, 2-5, 2-4, 2 or 3; e.g., one of m and n can be 0, and the other can be an integer from 1-9, e.g., 1-5, 1-4, 2-5, 2-4, 2 or 3; e.g., m+n=0);

R′ can be H, alkyl, alkenyl, alkynyl, cycloalkyl, heterocyclyl, aryl, or heteroaryl, each of which is optionally substituted (e.g., R′ can be hydrogen; optionally substituted alkyl, optionally substituted aryl; or heteroaryl). In certain embodiments, R′ can be hydrogen.

In embodiments, one of m and n can be 0. The other of m and n can be an integer from 1-9, e.g., 1-5, 1-4, 2-5, 2-4, 2 or 3 (e.g., 2-4). For example, n can be 0, and m can be an integer from 1-9, e.g., 1-5, 1-4, 2-5, 2-4, 2 or 3. As another example, m can be 0, and n can be an integer from 1-9, e.g., 1-5, 1-4, 2-5, 2-4, 2 or 3.

In embodiments, m+n=3. One of m and n can be 0 (e.g., n can be 0, and m can be 3 or vice versa).

In embodiments, m+n=2. One of m and n can be 0 (e.g., n can be 0, and m can be 2 or vice versa).

The compound can be 3,7,11,15-tetramethyl-1-hexadecen-3-ol.

The compound has the general formula:

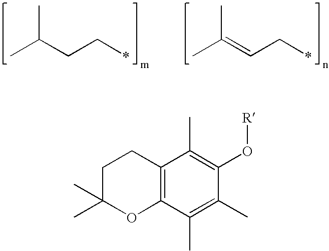

m+n=an integer from 0-9 (e.g., 0-5, 1-5, 2-5, 2-4, 2 or 3; e.g., one of m and n can be 0, and the other can be an integer from 1-9, e.g., 1-5, 1-4, 2-5, 2-4, 2 or 3; e.g., m+n=0);

R′ can be H, alkyl, alkenyl, alkynyl, cycloalkyl, heterocyclyl, aryl, or heteroaryl, each of which is optionally substituted (e.g., R′ can be hydrogen; optionally substituted alkyl, optionally substituted aryl; or heteroaryl, e.g., R′ can be hydrogen or optionally substituted alkyl). In certain embodiments, R′ can be optionally substituted alkyl.

In embodiments, one of m and n can be 0. The other of m and n can be an integer from 1-9, e.g., 1-5, 1-4, 2-5, 2-4, 2 or 3. For example, n can be 0, and m can be an integer from 1-9, e.g., 1-5, 1-4, 2-5, 2-4, 2 or 3. As another example, m can be 0, and n can be an integer from 1-9, e.g., 1-5, 1-4, 2-5, 2-4, 2 or 3.

In embodiments, m+n=3. One of m and n can be 0 (e.g., n can be 0, and m can be 3 or vice versa).

In embodiments, m+n=2. One of m and n can be 0 (e.g., n can be 0, and m can be 2 or vice versa).

R′ can be C₁-C₃ alkyl substituted with from 1-3 (e.g., 2) halogens, (e.g., fluoro (F).

The compound can be 6-Difluoromethoxy-2,5,7,8-tetramethyl-2-(4,8,12-trimethyl-tridecyl)-chroman.

The compound (e.g., a substantially pure compound) can be administered in the form of a composition, e.g., a pharmaceutical composition.

The compound can be administered intra-dermally, intra-peritoneally, orally, or intra-nasally.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-E set forth synthetic reaction schemes for using and preparing the compound classes described herein. The bolded number underneath a compound corresponds to the particular compound class, while R, R′, R″, and R′″ convey one or more isoprenoid units and/or one or more saturated isoprenoid units conjugated to one another.

FIG. 2 is a graph illustrating oxidative burst of granulocytes after in vitro stimulation with compound 1 (phytol) (♦),compound 2 (farnesol) (●), compound 3 (geranyl geraniol) (▴) or pristane (◯).

FIG. 3 is a bar graph demonstrating oxidative burst response to PMA stimulation in vitro in spleen granulocytes taken 5 days after s.c. injection of compound 1 (phytol) in DA.Ncf1^{DA} rats (200 μl) (Ph), compared to naive DA.Ncf1^{DA} and DA.Ncf1^{E3} (n=5).

FIG. 4 is a set of bar graphs illustrating the tissue distribution of 3H-labeled compound 1 (phytol) at day 2, 4, 8, 11, and 17 post-injection.

FIG. 5 is a graph demonstrating the severity of PIA in DA.Ncf1^{DA} rats treated with injection of compound 1 (phytol) (▴) compared with untreated DA.Ncf1^{DA} rats (Δ).*P<0.05, **P<0.01, ***P<0.001.

FIG. 6 is a set of graphs illustrating the effect of treatment of established PIA with compound 1 (phytol). FIG. 6A demonstrates the severity of arthritis in DA.Ncf1^{DA} rats after injections of compound 1 at the estimated peak of disease (day 22). (Significance indications above line represent s.c. administration and below i.p. administration) (control (Δ), compound 1 (phytol) i.p (◯), compound 1 (phytol) s.c. (▴)). *P<0.05, **P<0.01, ***P<0.001. FIG. 6B shows the effect of injections of compound 1 on serum levels of COMP (ng/ml). FIG. 6C demonstrates the severity of arthritis in DA.Ncf1^{DA} rats after injections of compound 1 in the chronic phase, (control (Δ) and compound 1 (phytol) s.c. (▴)), *P<0.05, **P<0.01, ***P<0.001. FIG. 6D demonstrates the effect of injections of compound 1 on the severity of arthritis in Lewis.IF rats (control (Δ) and compound 1 (phytol) s.c. (▴)), *P<0.05, **P<0.01, ***P<0.001. FIG. 6E demonstrates the effect injections of compound 1 on the severity of arthritis in rats with a functional oxidative burst (control (Δ) and compound 1 (phytol) (▴)), *P<0.05, **P<0.01, ***P<0.001.

FIG. 7 is a set of graphs illustrating the effect of compound 1 (phytol) on collagen induced arthritis in DA.Ncf1^{DA} rats. FIG. 7A demonstrates the severity of arthritis after s.c. administration of compound 1 (phytol) before immunization with CII (control (Δ) n=7, compound 1 (phytol) (▴) n=8). FIG. 7B shows the level of CII antibodies in serum.

FIG. 8 is a set of graphs illustrating the effect of compound 1 (phytol) on non oil collagen induced arthritis in DA.Ncf1^{DA} rats. FIG. 8A demonstrates the severity of arthritis after s.c. administration of compound 1 (phytol) before immunization with CII (n=7), (control (Δ) and compound 1 (phytol) (▴)). FIG. 8B shows the effect on the level of DTH response of phytol injection as measured by increase in ear volume (control n=7 and compound 1 (phytol) n=8). *P<0.05, **P<0.01, ***P<0.0011

FIG. 9 is a graph demonstrating the severity of PIA in DA.Ncf1^{DA} rats treated with injections of compound 1 (phytol) (♦) or compound 5 (phytyl acetate) (▴). Untreated control (◯).

FIG. 10 is a graph demonstrating the severity of PIA in DA.Ncf1^{DA} rats treated with injections of compound 1 (phytol) (♦) or compound 8 (▴). Untreated control (◯).

FIG. 11 is a graph demonstrating the severity of PIA in DA.Ncf1^{DA} rats treated with injections of compound 2 (farnesol) (▪) or compound 4 (farnesyl acetate) (▴). Untreated control (◯).

FIG. 12 is a graph demonstrating the severity of PIA in DA.Ncf1^{DA} rats treated with injections of compound 1 (phytol) (♦), compound 6 (di-hydro-phytol (▪), compound 5 (phytyl acetate) (▴) or compound 7 (di-hydro-phytyl acetate (●). Untreated control (◯).

FIG. 13 is a graph demonstrating the severity of PIA in DA.Ncf1^{DA} rats treated with oral administration of phytol 1% daily (7202244831), phytol 1% every 2'nd day (▴), or phytol 1% initiated day 7 after arthritis induction. Untreated control (◯), Omega3 fatty acid (▪).

FIG. 14 is a graph demonstrating the severity of PIA in DA.Ncf1^{DA} rats treated with oral administration of compound 1 (phytol) 1% daily (♦) or compound 5 (phytyl acetate) 1% daily (▴). Untreated control (◯).

FIG. 15 is a graph illustrating oxidative burst of granulocytes after in vitro stimulation with compound 1 (phytol) (♦), compound 2 (farnesol) (▴) or compound 9 (isophytol) (●).

FIG. 16 is a graph demonstrating the severity of PIA in DA.Ncf1^{DA} rats treated by oral gavage with compound 1 (phytol) 1.6 g/kg/every second day (♦), compound 1 (phytol) 3.2 g/kg/every second day (▴) or untreated control (◯).

FIG. 17 is a graph demonstrating the severity of PIA in DA.Ncf1^{DA} rats treated with oral administration of compound 1 (phytol) 500 mg/kg/day (♦), compound 9 (isophytol) 500 mg/kg/day (▴), or fenofibrate 100 mg/kg/day (□) or untreated control (◯).

FIG. 18 is a graph demonstrating the effect of compound 1 (phytol) on the development of EAE in DA.Ncf1^{DA} rats treated with parenteral administration of compound 1 (phytol) (♦) or untreated control (◯).

### DETAILED DESCRIPTION

The invention provides methods and materials related to treating autoimmune conditions (e.g., arthritis and multiple sclerosis). In particular, compounds that include one or more one or more isoprenoid units (e.g., one or more unsaturated isoprenoid units and/or one or more saturated isoprenoid units); compositions, e.g., pharmaceutical compositions, containing such compounds; and methods for using the same to treat or to alleviate the symptoms of autoimmune disease are described.

1. Compositions of Matter

The invention provides compounds useful for treating autoimmune diseases. In embodiments, the compounds can enhance NADPH oxidase activity. Pharmaceutically acceptable derivatives of any of the compounds described herein, e.g. compounds having any of the structural formulae delineated in compound classes 1-46, are also contemplated. Pharmaceutical compositions including any of the compounds described herein, e.g., in combination with a pharmaceutically acceptable carrier or diluent, are also contemplated.

As used herein, “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of an autoimmune condition, such as arthritis or multiple sclerosis, are ameliorated or otherwise beneficially altered. Treatment also encompasses any pharmaceutical use of the compounds or compositions herein, such as uses for treating diseases, disorders, or ailments in which an autoimmune condition is suspected or implicated, e.g., in a mammal such as a human.

As used herein, amelioration of the symptoms of a particular disorder by administration of a particular compound or pharmaceutical composition refers to any lessening, whether permanent or temporary, lasting or transient that can be attributed to or associated with administration of the composition.

In general, a compound includes one or more isoprenoid units, and more specifically one or more unsaturated isoprenoid units and/or one or more saturated isoprenoid units. As used herein, the term “isoprenoid unit” refers to a five carbon unit that is represented by the structure:

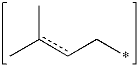

where [symbol] represents a single or a double bond.

The term “unsaturated isoprenoid unit” refers to a five carbon unit that is represented by the structure:

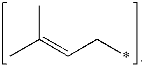

The term “saturated isoprenoid unit” refers to a five carbon unit that is represented by the structure:

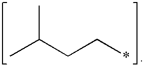

For purposes of clarification, the right most line (i.e., the line that extends rightward from the “head” of the isoprenoid unit and has the terminus labelled with an asterisk (*)) in each of the above three structural representations (as well as other such representations shown throughout the specification and claims) is intended to represent a covalent bond that can occur (i) between the “head” terminus of said isoprenoid unit and, e.g., a tail or head terminus of another unsaturated or saturated isoprenoid unit; or (ii) between the “head” terminus of said isoprenoid unit and an atom that forms part of another structural moiety, e.g. a functional moiety. I.e., the terminus marked with the asterisk (*) in the aforementioned structures is not intended to show a sixth carbon atom attached to the head of said isoprenoid unit.

By way of example, a compound that contains a saturated isoprenoid unit that is directly connected, in head to tail fashion, to an unsaturated isoprenoid unit would have the following structure: 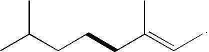
The bolded line in the above 10-carbon structure represents the covalent bond between the head terminus of the saturated isoprenoid unit and the tail terminus of the unsaturated isoprenoid unit.

In some embodiments, the unsaturated isoprenoid and/or saturated isoprenoid units can be conjugated (e.g., covalently bound) to one another. Thus, as used herein, brackets around an unsaturated isoprenoid and/or saturated isoprenoid unit structure represent the ability of a unit to be conjugated at either the head or tail termini, e.g., to another unit. In certain embodiments, a compound can include up to 10 (e.g., 1, 2, 3, 4, 5, 6, 7, 8, 9, or 10, e.g., 2, 3, or 4, e.g., 2 or 3, e.g. 3) unsaturated isoprenoid units and/or saturated isoprenoid units conjugated to one another. A compound can include unsaturated isoprenoid units and/or saturated isoprenoid units arranged in any sequence or directionality relative to one another.

In some embodiments, one or more of the unsaturated isoprenoid units and/or saturated isoprenoid units can be derivatized with one or more functional moieties. A compound can include derivatized unsaturated isoprenoid units and/or derivatized saturated isoprenoid units arranged in any sequence or directionality relative to one another and in any sequence or directionality relative to underivatized unsaturated isoprenoid units and/or underivatized saturated isoprenoid units. Thus, a derivatized unsaturated isoprenoid unit or a derivatized saturated isoprenoid unit can be at any position in a compound, e.g., at an internal or at a terminal unit. For example, a compound can include a saturated isoprenoid unit conjugated to an unsaturated isoprenoid unit, which is in turn conjugated to a derivatized (saturated or unsaturated) isoprenoid unit. In another example, a compound can include three saturated isoprenoid units, with the second saturated isoprenoid unit conjugated to the third derivatized saturated isoprenoid unit.

In some embodiments, a functional moiety can be conjugated (e.g., covalently bound) to an unsaturated isoprenoid unit and/or saturated isoprenoid unit at any carbon position in the unit, e.g., at a terminal, branching, or internal carbon atom of the unit. A functional moiety can comprise any functional group, including, without limitation, alkyl, alkenyl, alkynyl, cycloalkyl, alcohol, ketone, aldehyde, carboxylic acid, carboxylic acid ester, thio ester, carbamate, halide, alkyl ether, aryl ether, thio ether, amine, amide, heterocyclic (e.g., piperazinyl), heteroaryl (thienyl, furyl, pyridyl) or aryl (e.g., phenyl or naphthyl) groups, or combinations thereof.

The term “alkyl” refers to a saturated hydrocarbon chain that may be a straight chain or branched chain, containing the indicated number of carbon atoms. For example, the term “C₁₋₂₀-alkyl” denotes a straight or branched alkyl group having from 1 to 20 carbon atoms. Examples of alkyl include methyl, ethyl, n-propyl, iso-propyl, n-butyl, iso-butyl, sec-butyl, t-butyl and straight- and branched-chain pentyl and hexyl. For parts of the range “C₁₋₂₀-alkyl” all subgroups thereof are contemplated (e.g., C₁-C₁₂, C₁-C₁₀, C₁-C₆, or C₁-C₃ linear or branched saturated chains).

The term “alkenyl” refers to a straight or branched hydrocarbon chain containing 2-20 carbon atoms and having one or more double bonds. The term “alkynyl” refers to a straight or branched hydrocarbon chain containing 2-20 carbon atoms and having one or more triple bonds. Examples of said alkenyl include vinyl, allyl, 1-butenyl, 1-pentenyl, and 1-hexenyl. Examples of alkynyl include ethynyl, propargyl, and 3-hexynyl. For parts of the ranges “C₂₋₂₀-alkenyl” and “C₂₋₂₀-alkynyl,” all subgroups thereof are contemplated (e.g., C₂-C₁₂, C₂-C₁₀, C₂-C₆, or C₂-C₄ linear or branched).

The term “cycloalkyl” refers to saturated monocyclic, bicyclic, tricyclic, or other polycyclic hydrocarbon C₃-C₂₀ (e.g., C₃-C₁₀, C₃-C₈, C₃-C₆) rings. Cycloalkyl groups can contain fused rings. Fused rings are rings that share a common carbon atom. Cycloalkyl moieties can include, e.g., cyclopropyl, cyclobutyl, cyclopentyl, cyclohexyl, cycloheptyl, adamantyl, and norbornyl (bicycle[2.2.1]heptyl).

Alkyl, alkenyl, alkynyl, and cycloalkyl groups can be optionally substituted (e.g., with from 1-5 (e.g., 1-4, 1-3, 1-2, or 1) substituents, e.g., X (e.g., F, Cl, Br, I), NH₂, NO₂, CN, OH, alkyl, cycloalkyl, or carboxylic acid ester moieties (or any combination thereof when more than one substituent is present).

The term “aryl” refers to a hydrocarbon ring System having at least one aromatic ring. Examples of aryls are phenyl, pentalenyl, indenyl, indanyl, isoindolinyl, chromanyl, naphthyl, fluorenyl, anthryl, phenanthryl and pyrenyl. Aryl groups may be substituted optionally with one or more (e.g., with from 1-5 (e.g., 1-4, 1-3, 1-2, or 1) substituents such as X (e.g., F, Cl, Br, I), NH₂, NO₂, CN, OH, alkyl (e.g., CH₃), or carboxylic acid ester moieties (or any combination thereof when more than one substituent is present).

The term “heteroaryl” means in the present description a monocyclic, bi- or tricyclic aromatic ring System (only one ring need to be aromatic) having from 5 to 14, preferably 5 to 10 ring atoms such as 5, 6, 7, 8, 9 or 10 ring atoms (mono- or bicyclic), in which one or more of the ring atoms are other than carbon, such as nitrogen, sulfur, oxygen and selenium as part of the ring System. Examples of such heteroaryl rings are pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline (i e 2,3-dihydroindole), isoindoline (i e 1,3-dihydroisoindole), benzothiophene, benzofuran, 2,3-dihydrobenzofuran, isobenzofuran, benzodioxole, benzothiadiazole, benzotriazole, benzoxazole, 2,1,3-benzoxadiazole, benzopyrazole, 2,1,3-benzothiazole, 2,1,3-benzoselenadiazole, benzimidazole, indazole, benzodioxane, 2,3-dihydro-1,4-benzodioxine, indane, 1,2,3,4-tetrahydroquinoline, 3,4-dihydro-2H-1,4-benzoxazine, 1,5-naphthyridine, 1,8-naphthyridine, pyrido[3,2-b]thiophene, acridine, fenazine and xanthene. Heteroaryl groups may be substituted optionally with one or more(e.g., with from 1-5 (e.g., 1-4, 1-3, 1-2, or 1) substituents such as X (e.g., F, Cl, Br, I), NH₂, NO₂, CN, OH, alkyl (e.g., CH₃), or carboxylic acid ester moieties (or any combination thereof when more than one substituent is present).

The term “heterocyclic” and “heterocyclyl” in the present description is intended to include unsaturated as well as partially and fully saturated mono-, bi- and tricyclic rings having from 4 to 14, preferably 4 to 10 ring atoms having one or more heteroatoms (e.g., oxygen, sulfur, or nitrogen) as part of the ring System and the reminder being carbon, such as, for example, the heteroaryl groups mentioned above as well as the corresponding partially saturated or fully saturated heterocyclic rings. Exemplary saturated heterocyclic rings are azetidine, pyrrolidine, piperidine, piperazine, morpholine, thiomorpholine, 1,4-oxazepane, azepane, phthalimide, indoline, isoindoline, 1,2,3,4-tetrahydroquinoline, 1,2,3,4-tetrahydroisoquinoline, hexahydroazepine, 3,4-dihydro-2(1H)isoquinoline, 2,3-dihydro-1H-indole, 1,3-dihydro-2H-isoindole, azocane, 1-oxa-4-azaspiro[4.5]dec-4-ene, decahydroisoquinoline, 1,2-dihydroquinoline, and 1,4-diazepane. Heterocyclic groups groups may be substituted optionally with one or more(e.g., with from 1-5 (e.g., 1-4, 1-3, 1-2, or 1) substituents such as X (e.g., F, Cl, Br, I), NH₂, NO₂, CN, OH, alkyl (e.g., CH₃), or carboxylic acid ester moieties (or any combination thereof when more than one substituent is present).

Generic formulae for particular classes of compounds are described below and can include one or more of the features described herein. Representative species for each generic formula are also set forth.

Compound class 1 includes branched alcohols having the general formula:

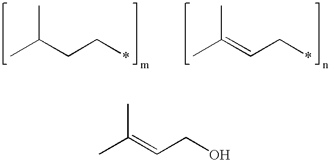

where n+m is an integer from 0 to 9. Particular species include:

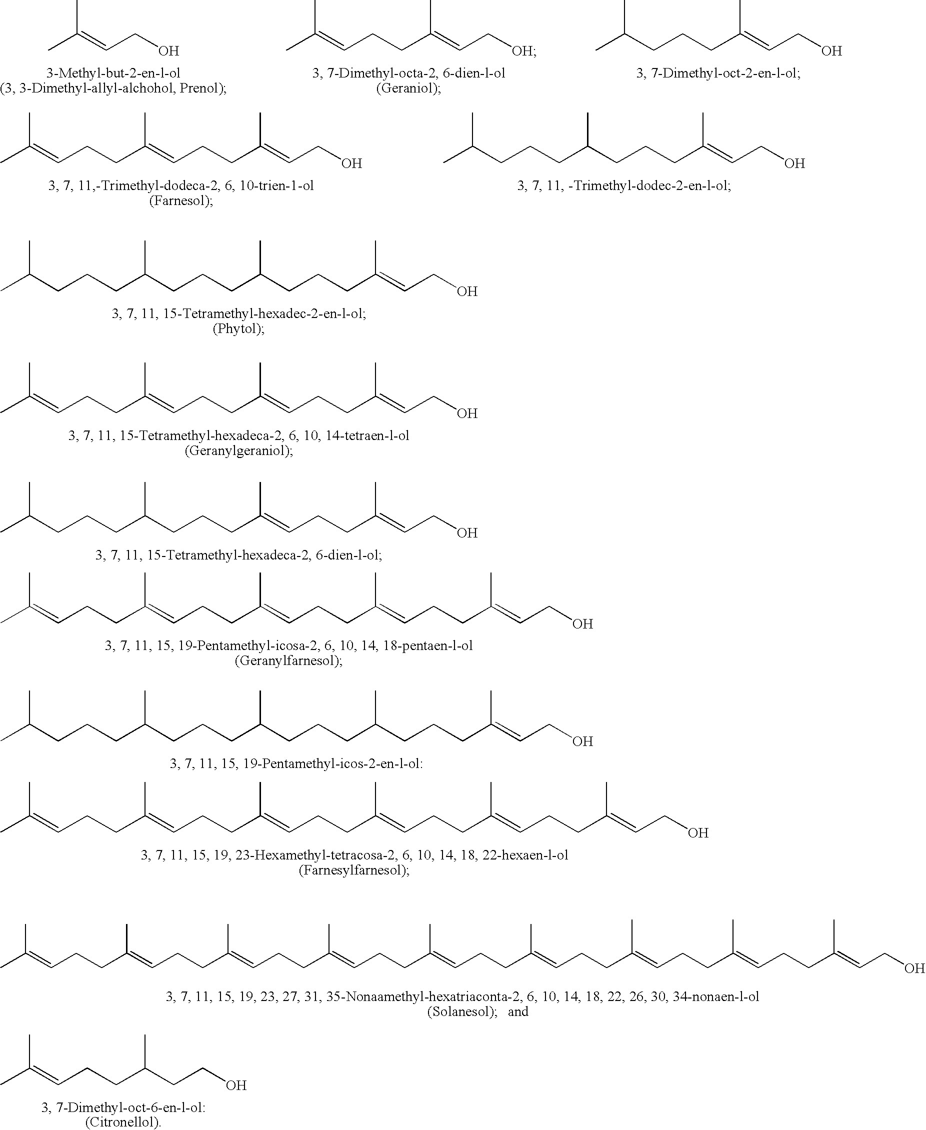

Compound class 2 includes carboxylic acid esters having the general formula:

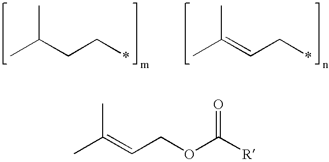

where n+m is an integer from 0 to 9; and where R′ can be, e.g., H, alkyl, alkenyl, alkynyl, cycloalkyl, heterocyclyl, aryl, or heteroaryl, each of which is optionally substituted. Particular compounds include:

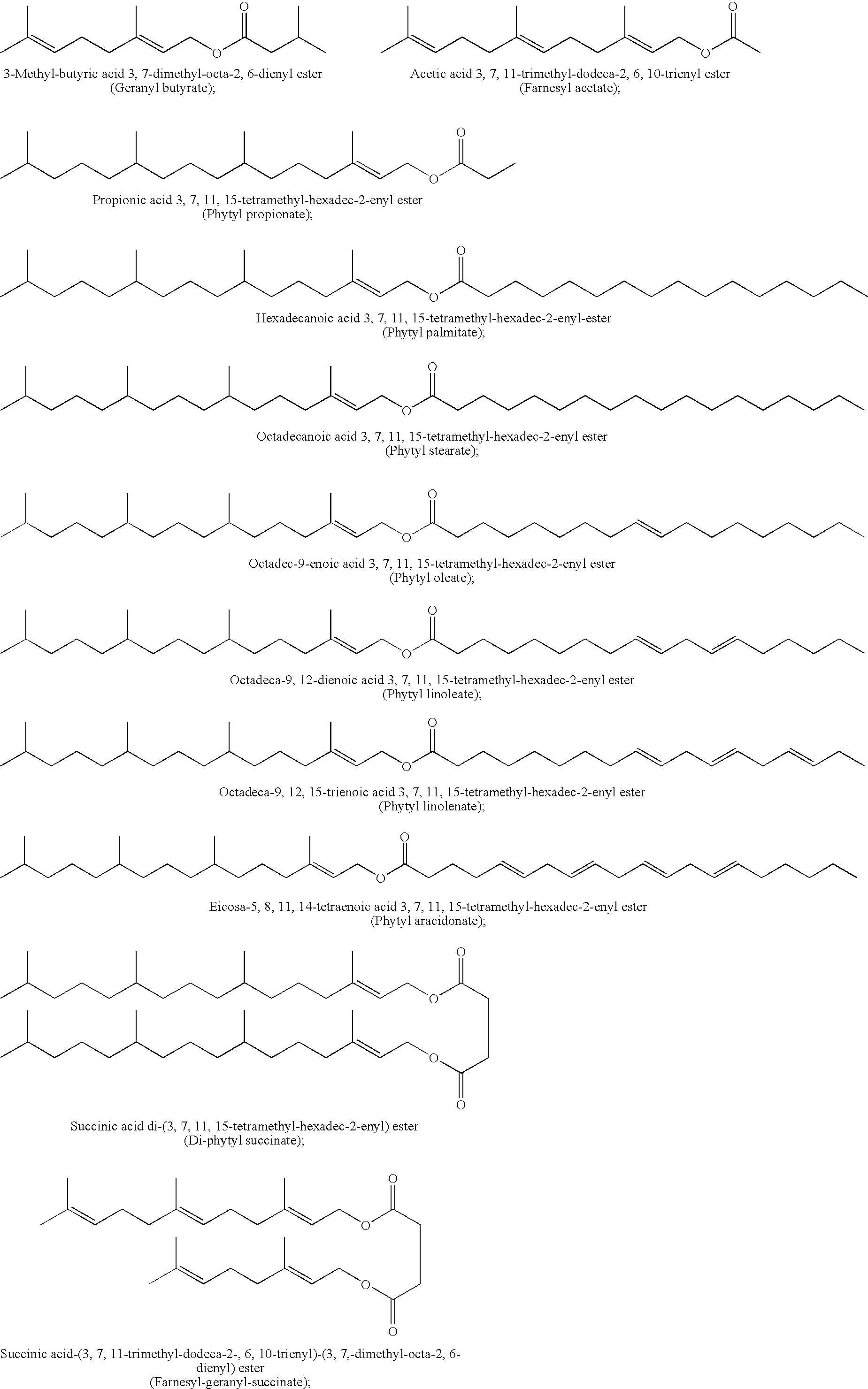

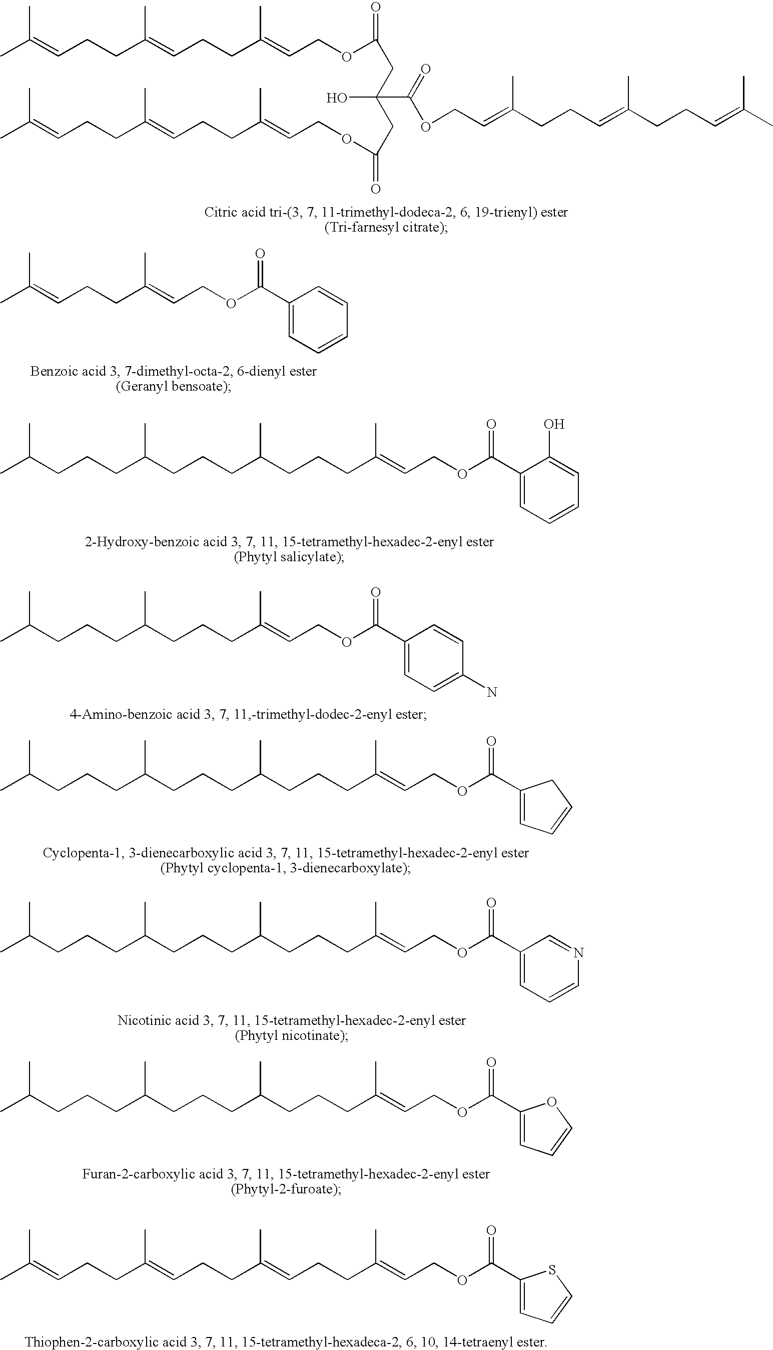

Compound class 3 includes carbamates with the general formula:

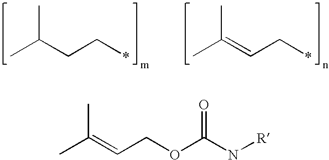

where n+m can be an integer from 0 to 9; and where R′ is, e.g., H, alkyl, alkenyl, alkynyl, cycloalkyl, heterocyclyl, aryl, or heteroaryl, each of which is optionally substituted. Particular examples include:

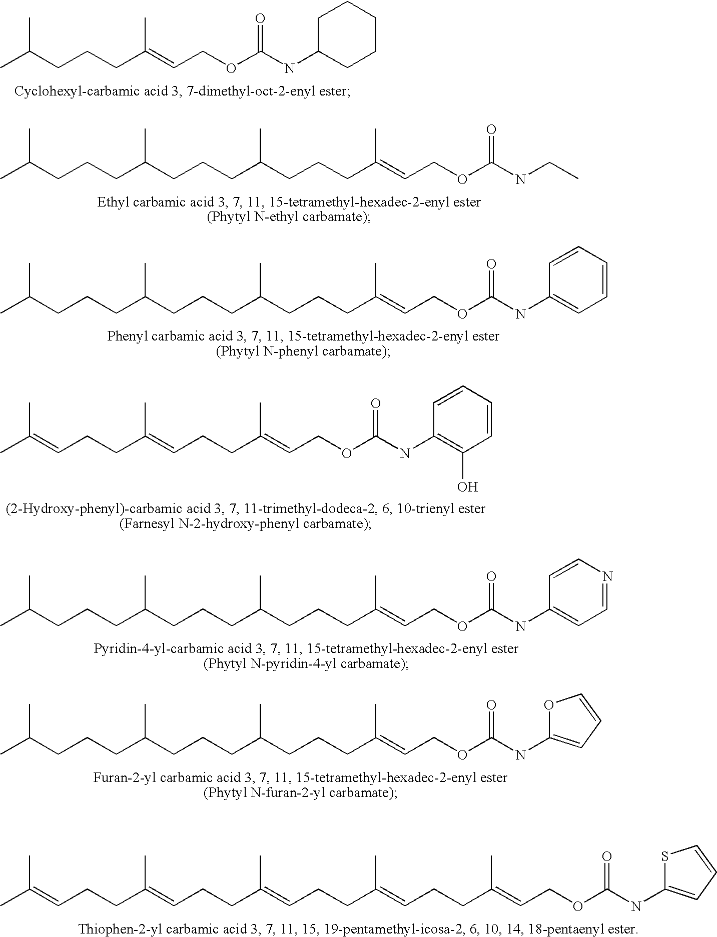

Compound class 4 substitutes a halogen moiety for the hydroxy moiety in composition class 1. Halogen moieties include F, Cl, Br, and I.

Compound class 5 includes aryl ethers with the general formula:

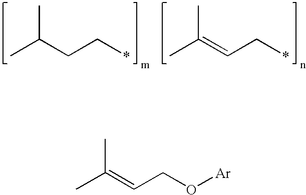

where n+m is an integer from 0 to 9; and Ar is optionally substituted aryl (e.g., phenyl). These compounds are exemplified by:

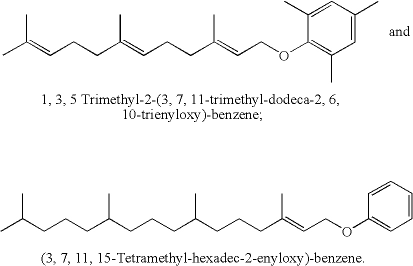

Compound class 6 includes thio ethers with the general formula:

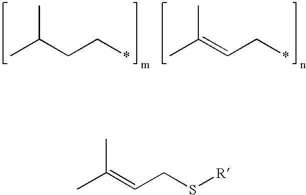

where n+m is an integer from 0 to 9; and where R′ is, e.g., H, alkyl, alkenyl, alkynyl, cycloalkyl, heterocyclyl, aryl, or heteroaryl, each of which is optionally substituted. Particular examples include:

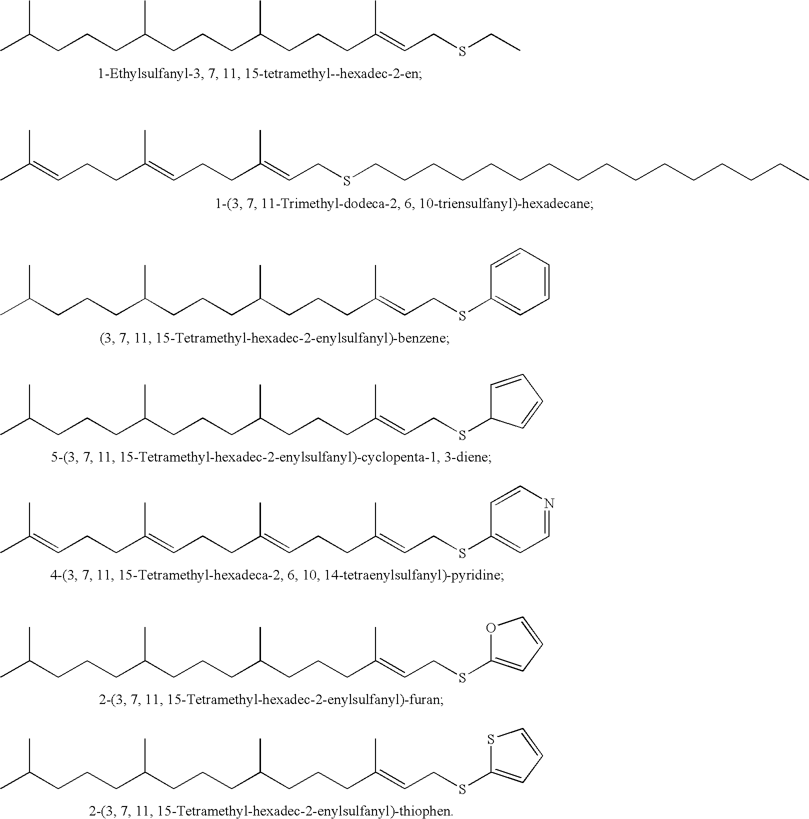

Compound class 7 include amines with the general formula:

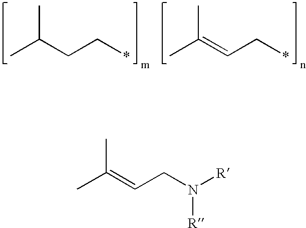

where n+m=an integer from 0 to 9; and where R′ and R″ independently are, e.g., H, alkyl, alkenyl, alkynyl, cycloalkyl, heterocyclyl, aryl, or heteroaryl, each of which is optionally substituted. Examples include:

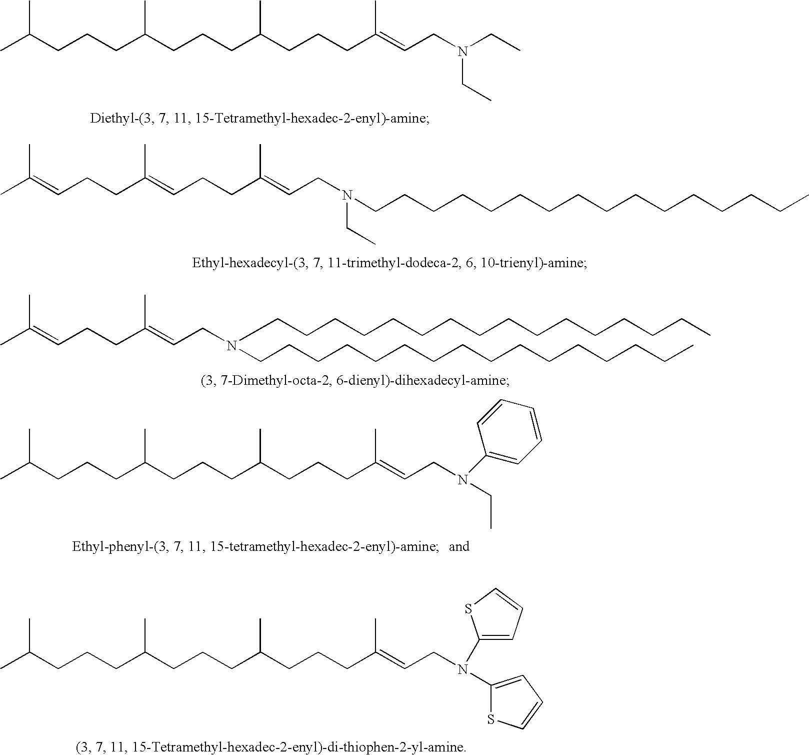

(3,7,11,15-Tetramethyl-hexadec-2-enyl)-di-thiophen-2-yl-amine.

Compound class 8 includes compounds with the general formula:

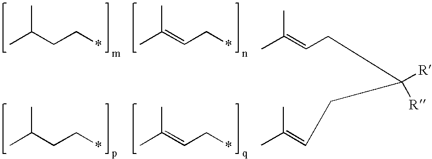

where (n+m) and (p+q) independently are an integer from 0 to 9; and R′ and R″ independently are, e.g., H, alkyl, alkenyl, alkynyl, cycloalkyl, heterocyclyl, aryl, or heteroaryl, each of which is optionally substituted. Examples include:

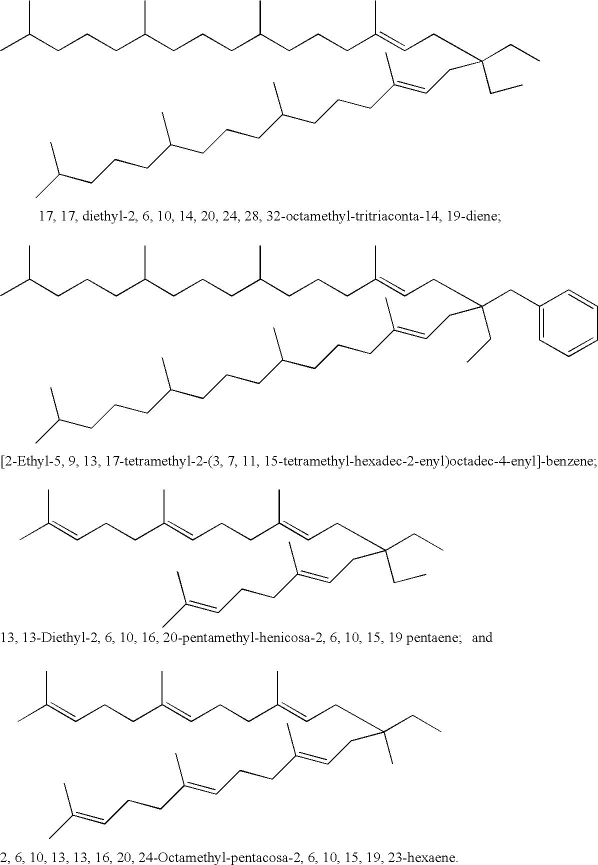

Compound class 9 includes compounds having the general formula:

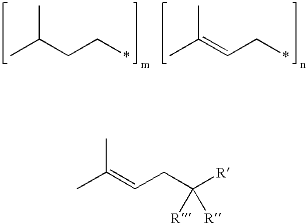

where n+m=an integer from 0 to 9; and R′, R″ and R′″ independently are e.g., H, alkyl, alkenyl, alkynyl, cycloalkyl, heterocyclyl, aryl, or heteroaryl, each of which is optionally substituted. Examples include:

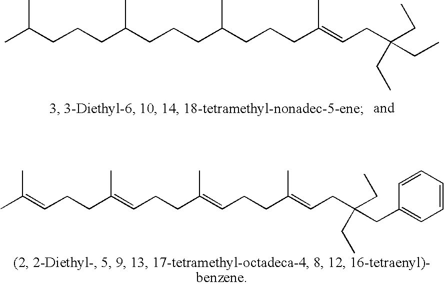

Compound class 10 includes carboxylic acids with the general formula:

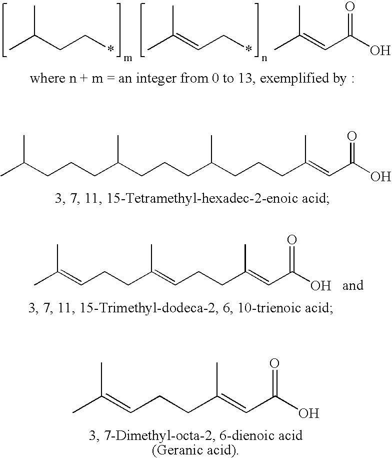

Compound class 11 includes amides of the general formula:

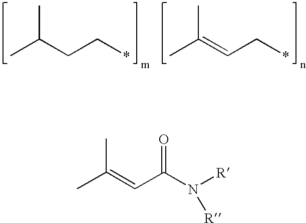

where n+m=an integer from 0 to 9; and R′ and R″ independently are, e.g., H, alkyl, alkenyl, alkynyl, cycloalkyl, heterocyclyl, aryl, or heteroaryl, each of which is optionally substituted. Examples include:

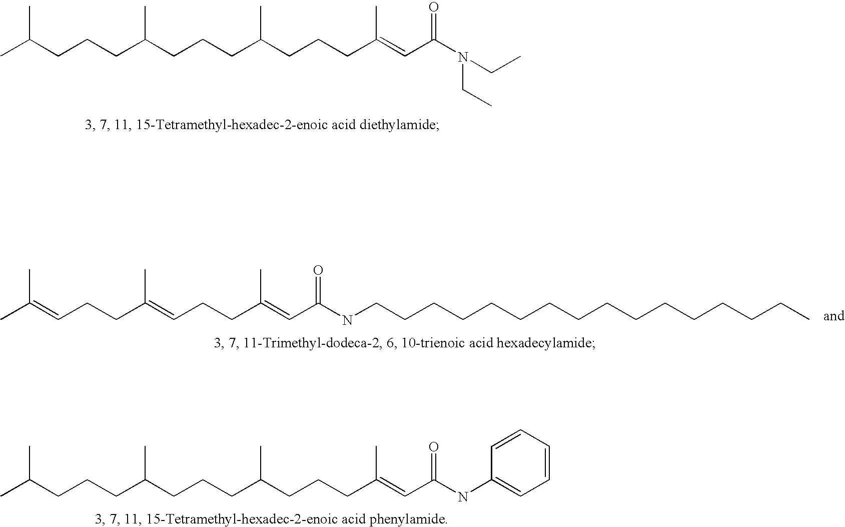

Compound class 12 includes carboxylic acid esters with the general formula:

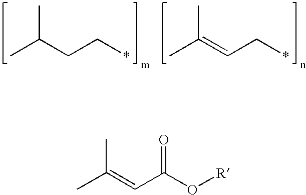

where n+m=an integer from 0 to 9; and R′ is alkyl, aryl, or heteroaryl. Examples include:

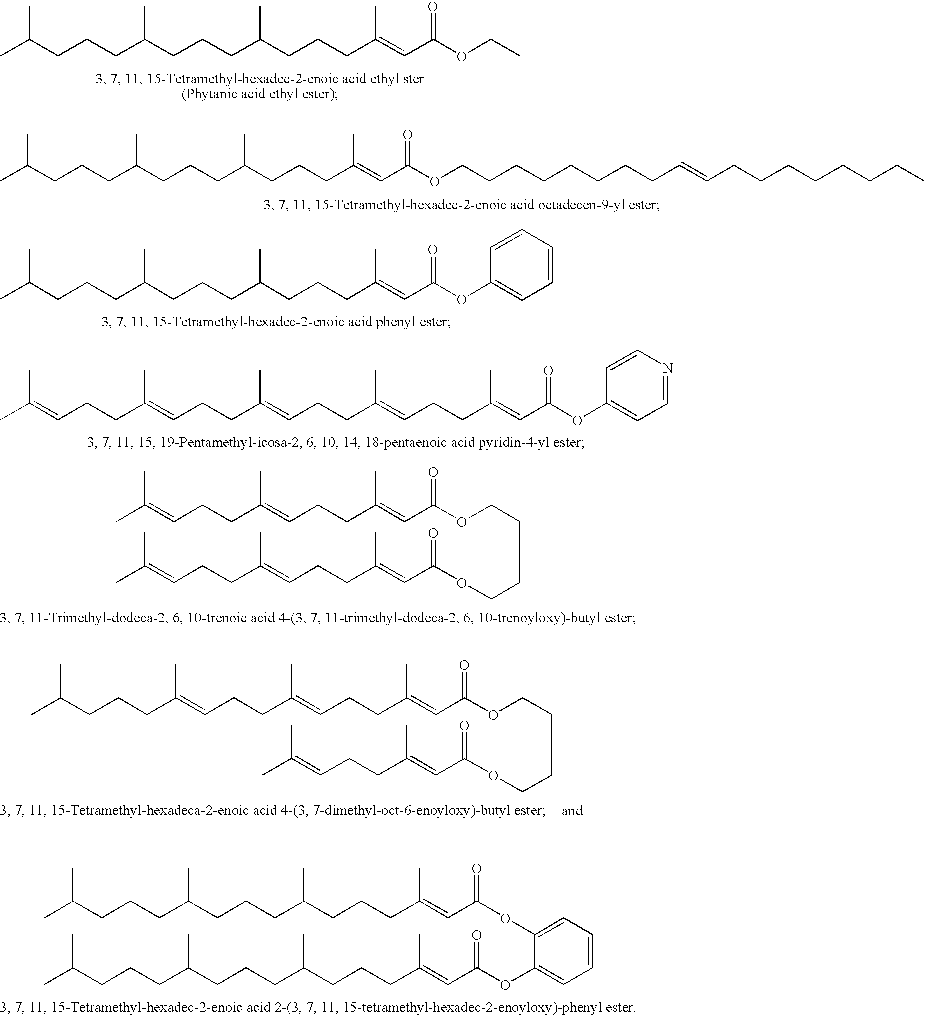

Compound class 13 includes branched alcohols with the general formula:

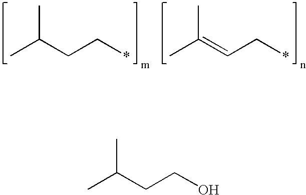

where n+m=an integer from 0 to 9. Examples include:

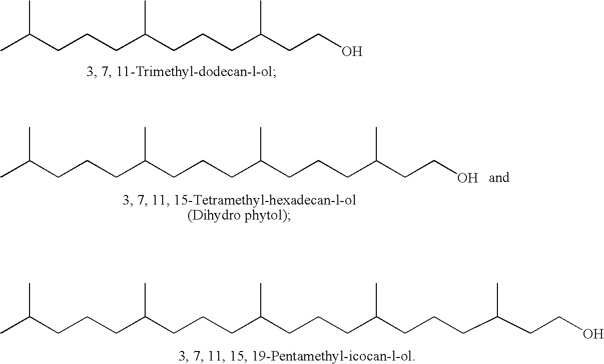

Compound class 14 includes carboxylic acid esters with the general formula:

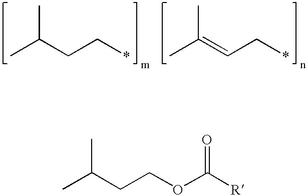

where n+m=an integer from 0 to 9; and where R′ is, e.g., H, alkyl, alkenyl, alkynyl, cycloalkyl, heterocyclyl, aryl, or heteroaryl, each of which is optionally substituted. Examples include:

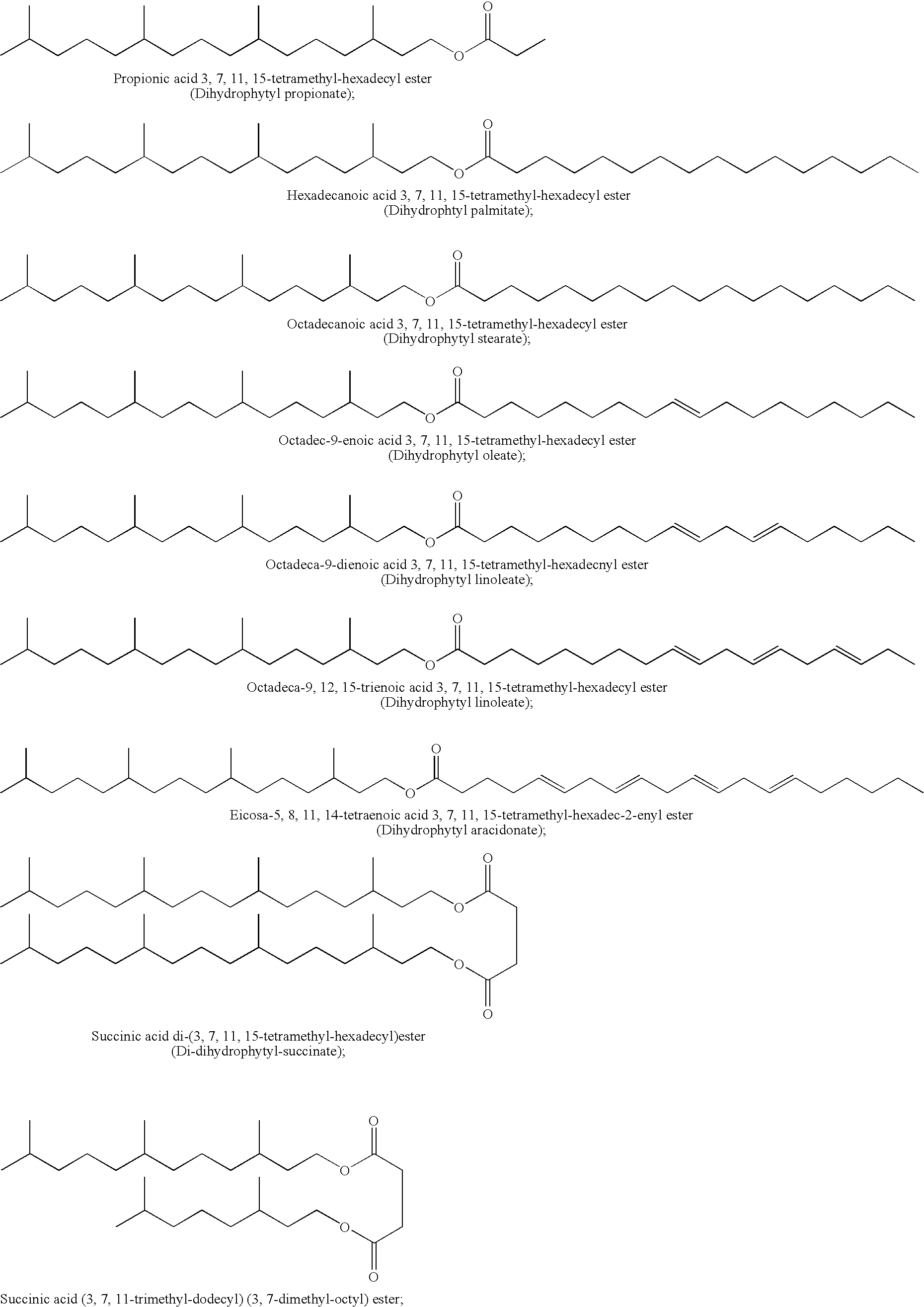

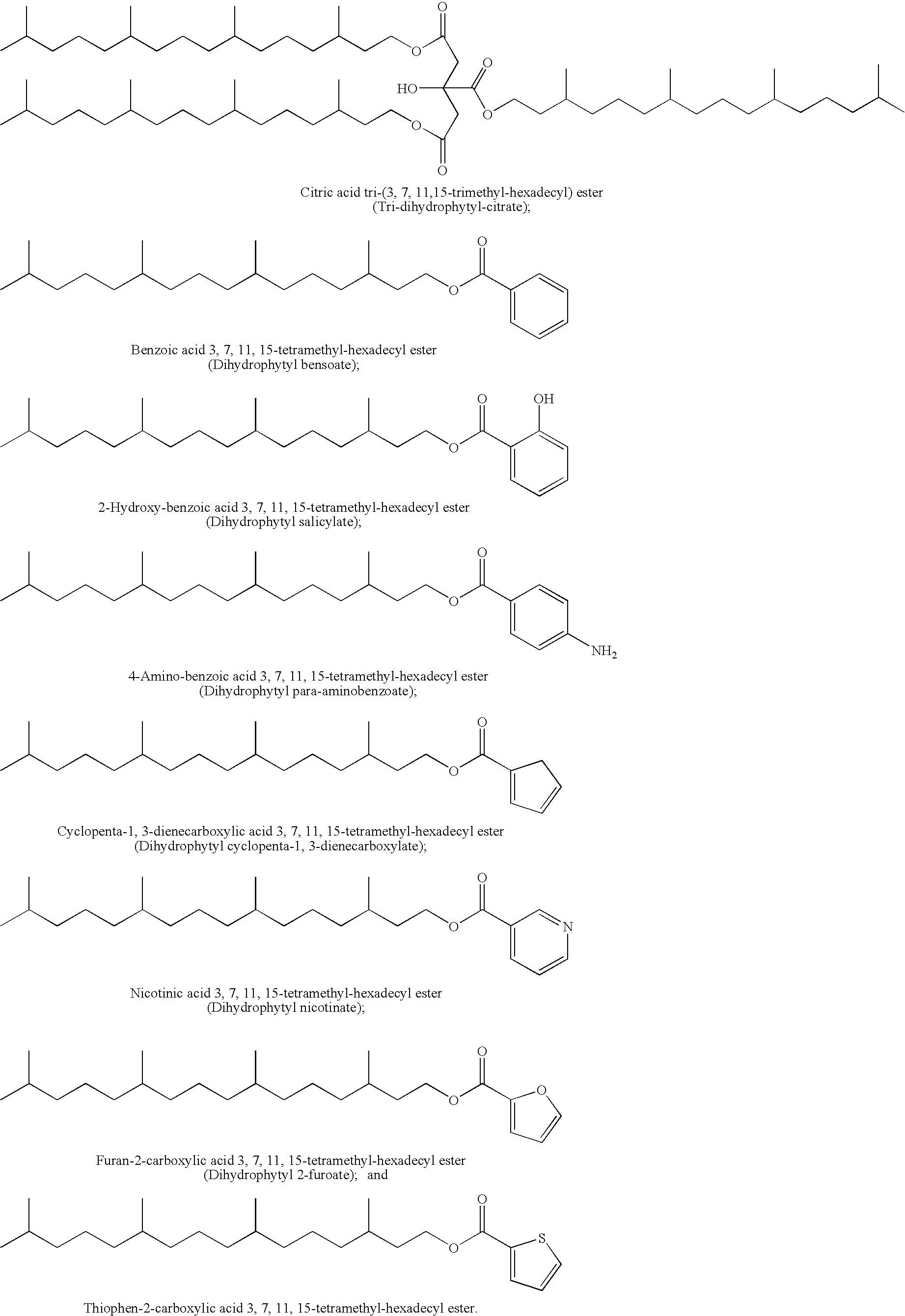

Compound class 15 includes carbamates with the general formula:

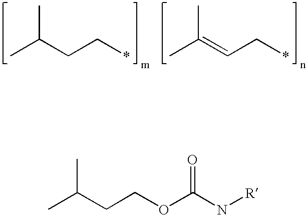

where n+m=an integer from 0 to 9; and R′ is, e.g., H, alkyl, alkenyl, alkynyl, cycloalkyl, heterocyclyl, aryl, or heteroaryl, each of which is optionally substituted. Examples include:

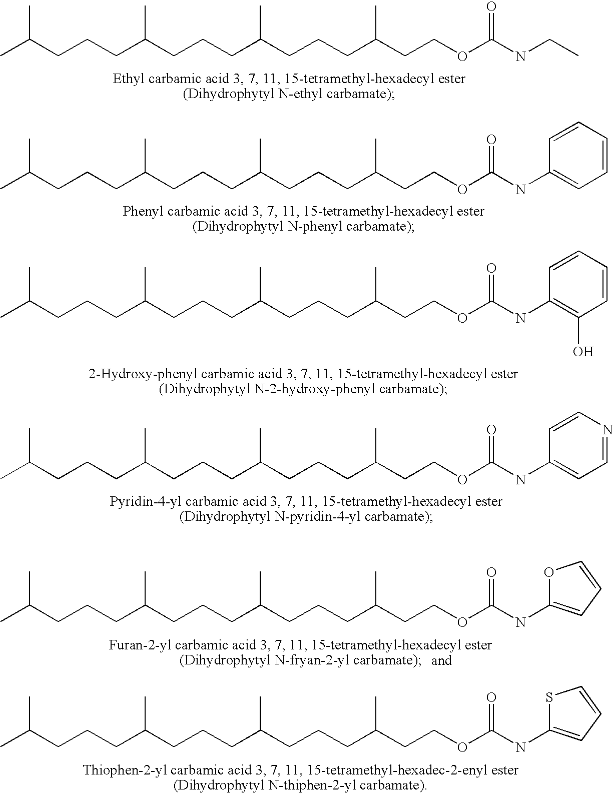

Compound class 16 substitutes a halogen moiety for the hydroxy moiety in compound class 13. Halogen moieties include F, Cl, Br, and I.

Compound class 17 includes aryl ethers with the general formula:

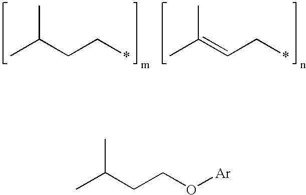

where n+m=an integer from 0 to 9; and Ar is optionally substituted aryl (e.g., phenyl), exemplified by:

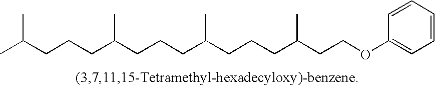

Compound class 18 includes thio ethers with the general formula:

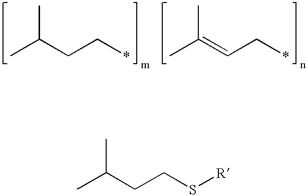

where n+m=an integer from 0 to 9; and where R′ is, e.g., H, alkyl, alkenyl, alkynyl, cycloalkyl, heterocyclyl, aryl, or heteroaryl, each of which is optionally substituted. Examples include:

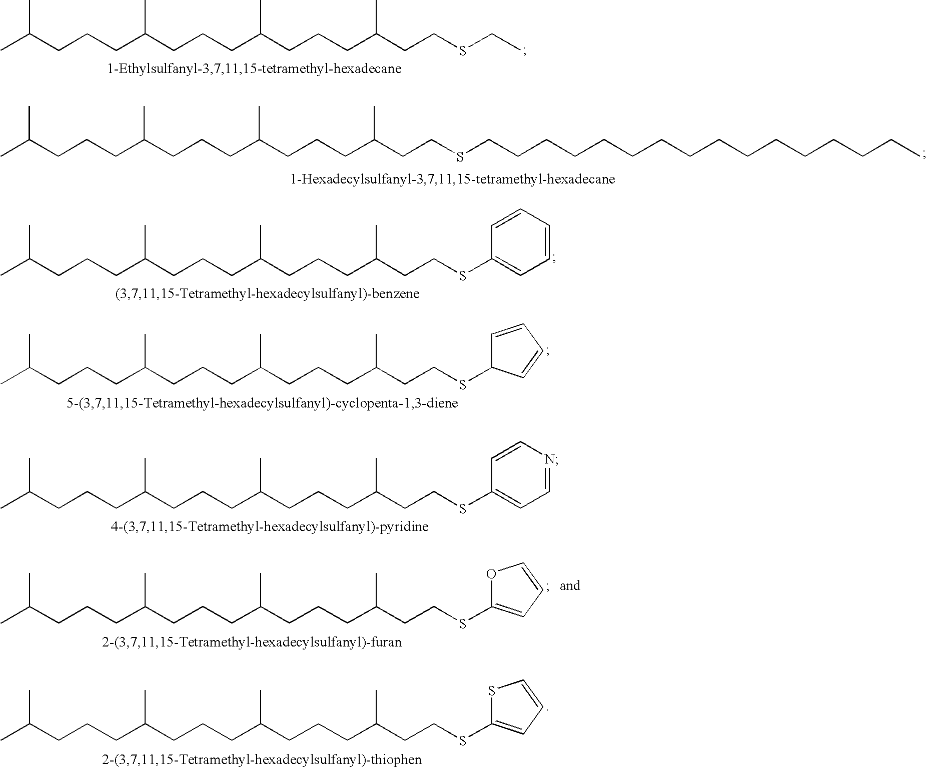

Compound class 19 includes amines with the general formula:

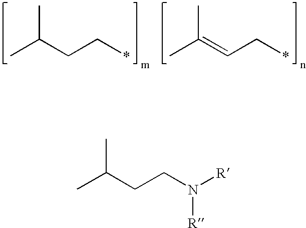

where n+m=an integer from 0 to 9; and where R′ and R″ independently are, e.g., H, alkyl, alkenyl, alkynyl, cycloalkyl, heterocyclyl, aryl, or heteroaryl, each of which is optionally substituted. Some examples include:

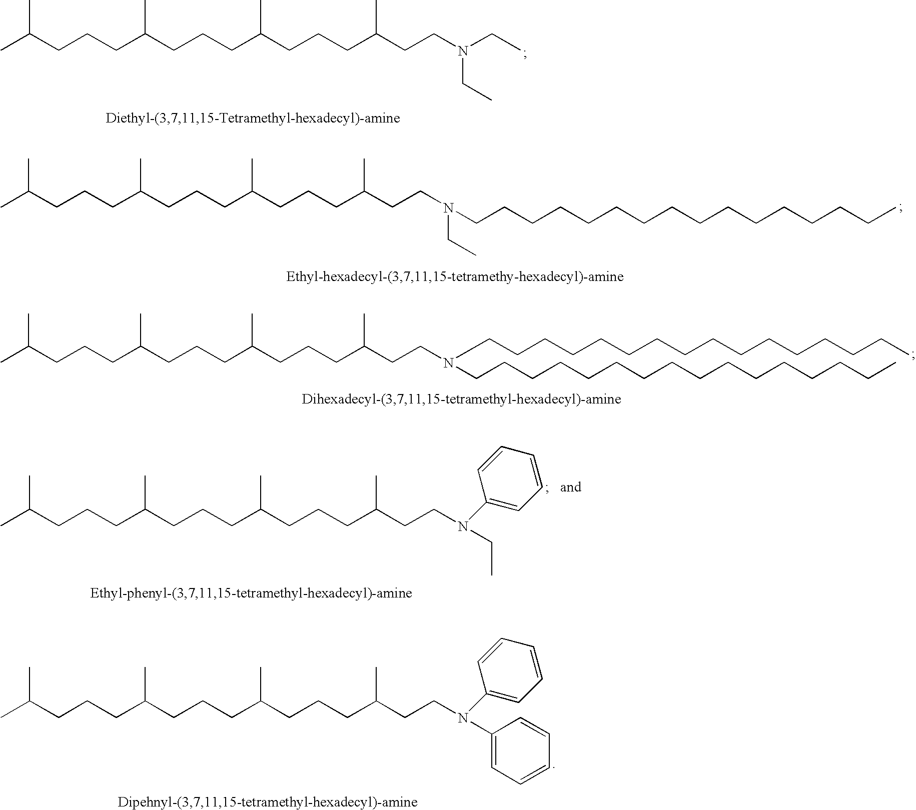

Compound class 20 includes compounds with the general formula:

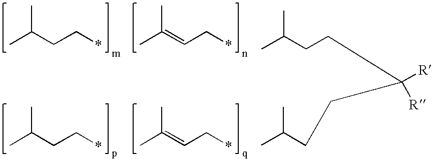

where n+m=an integer from 0 to 9; p+q=an integer from 0 to 9; and R′ and R″ independently are H, alkyl, alkenyl, alkynyl, cycloalkyl, heterocyclyl, aryl, or heteroaryl, each of which is optionally substituted. Examples include:

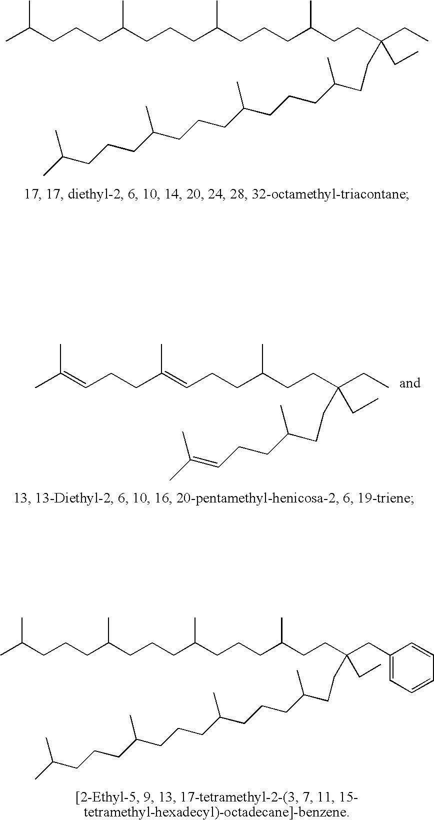

Compound class 21 includes compounds with the general formula:

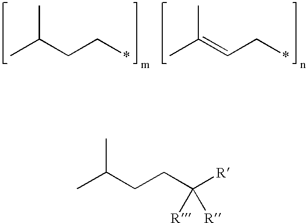

where n+m=an integer from 0 to 9; and R′, R″ and R′″ independently are, e.g., H, alkyl, alkenyl, alkynyl, cycloalkyl, heterocyclyl, aryl, or heteroaryl, each of which is optionally substituted, exemplified by:

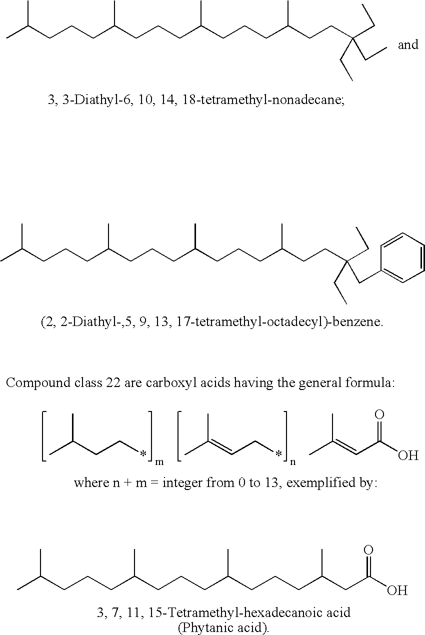

Compound class 23 includes amides of the general formula:

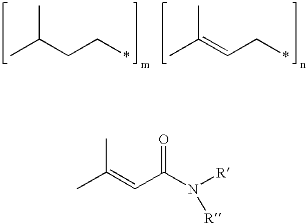

where n+m=an integer from 0 to 9; and R′ and R″ independently are, e.g., H, alkyl, alkenyl, alkynyl, cycloalkyl, heterocyclyl, aryl, or heteroaryl, each of which is optionally substituted. Examples include:

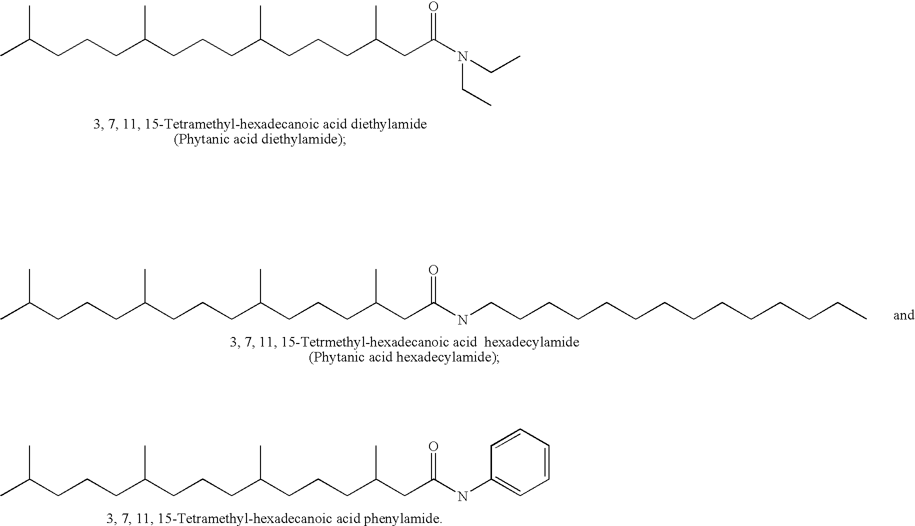

Compound class 24 includes carboxylic acid esters with the general formula:

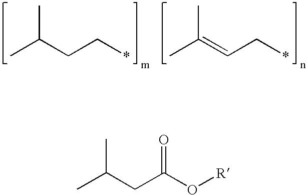

where n+m=an integer from 0 to 9; and R′ is, e.g., alkyl, alkenyl, alkynyl, cycloalkyl, heterocyclyl, aryl, or heteroaryl, each of which is optionally substituted, exemplified by:

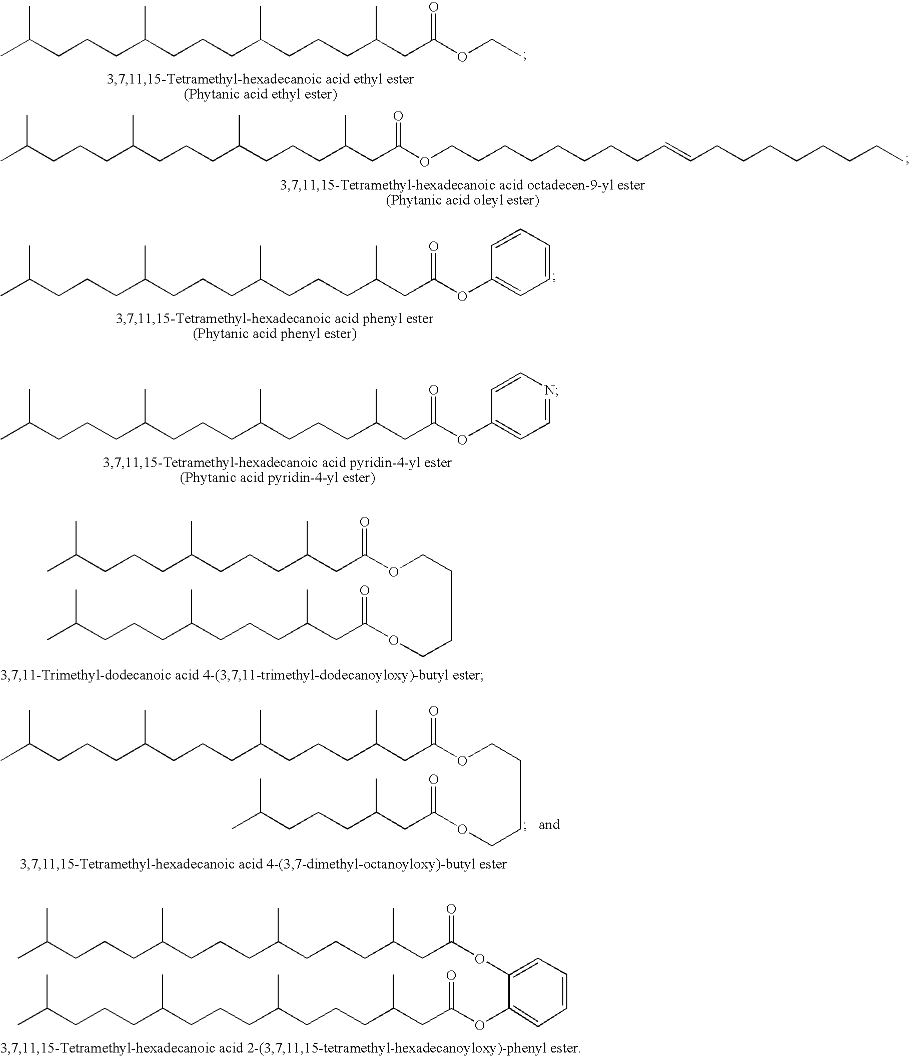

Compound class 25 includes compounds with the general formula:

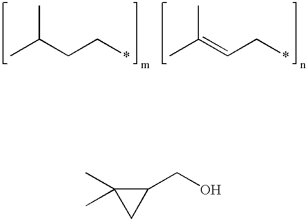

where n+m=an integer from 0 to 9, exemplified by:

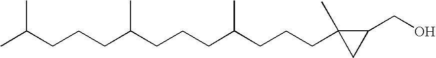

Compound class 26 includes compounds with the general formula:

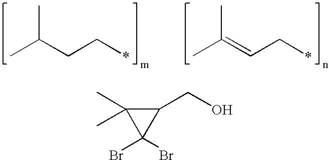

where n+m=an integer from 0 to 9, exemplified by:

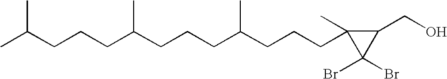

[2,2-Dibromo-3-methyl-3-(4,8,12-trimethyl-tridecyl)-cyclopropyl]-methanol.

Compound class 27 includes compounds with the general formula:

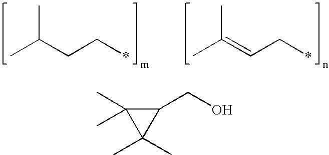

where n+m=an integer from 0 to 9, exemplified by:

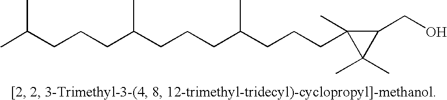

Compound class 28 includes compounds with the general formula:

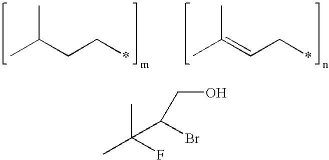

where n+m=an integer from 0 to 9, exemplified by:

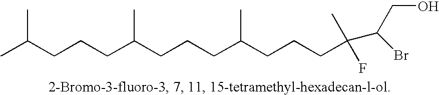

Compound class 29 are compounds with the general formula:

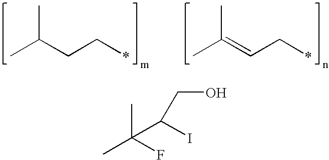

where n+m=an integer from 0 to 9, exemplified by:

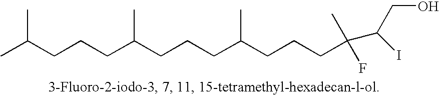

Compound class 30 include compounds having the general formula:

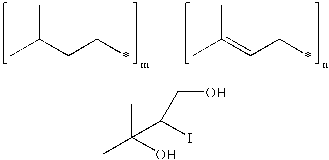

where n+m=an integer from 0 to 9, exemplified by:

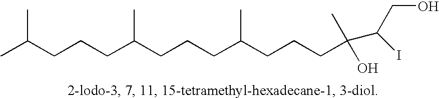

Compound class 31 includes compounds with the general formula:

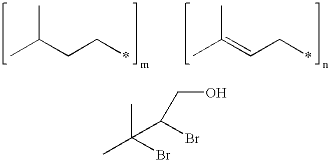

where n+m=an integer from 0 to 9, exemplified by:

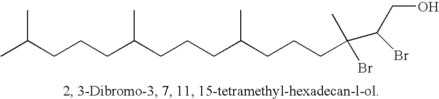

Compound class 32 are compounds with the general formula:

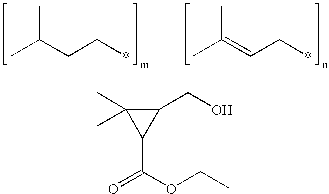

where n+m=an integer from 0 to 9, exemplified by:

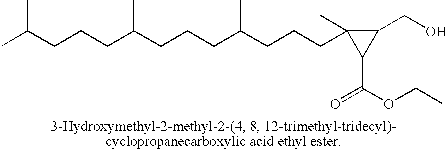

Compound class 33 includes compounds with the general formula:

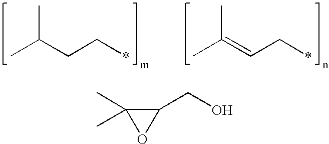

where n+m=an integer from 0 to 9, exemplified by:

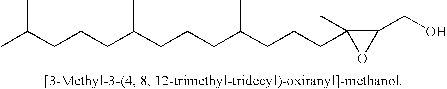

Compound class 34 are compounds having the general formula:

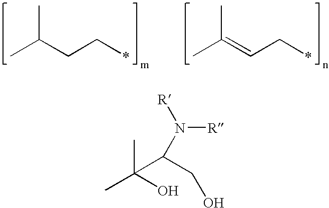

where n+m=an integer from 0 to 9; and where R′ and R″ independently are, e.g., H, alkyl, alkenyl, alkynyl, cycloalkyl, heterocyclyl, aryl, or heteroaryl, each of which is optionally substituted. Examples include:

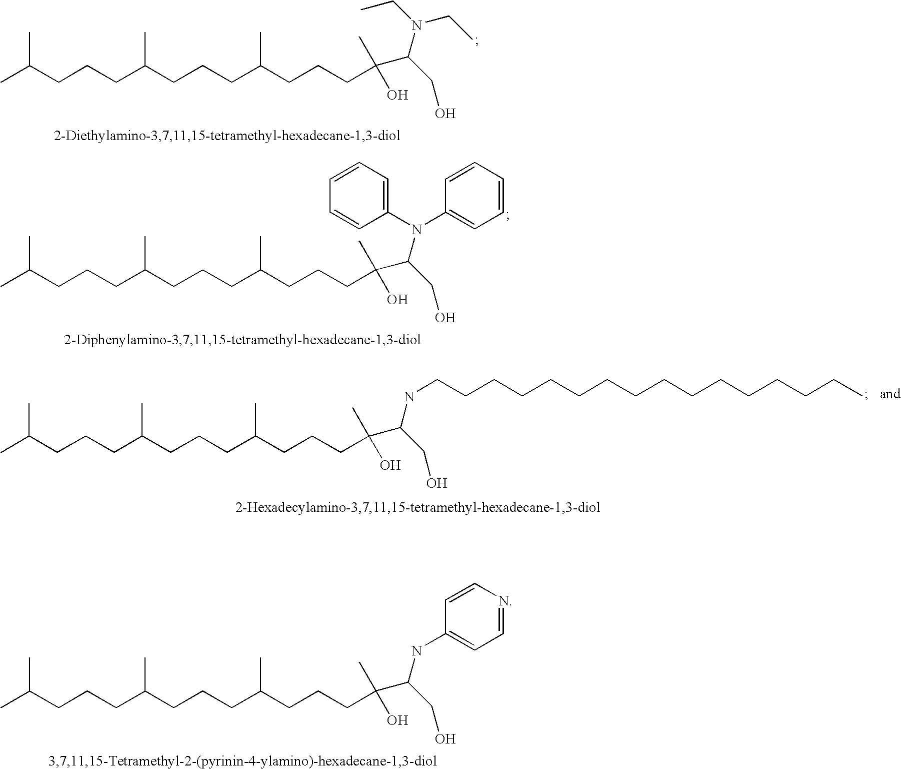

Compound class 35 are compounds with the general formula:

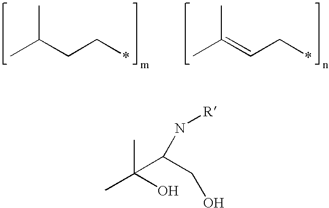

where n+m=an integer from 0 to 9; and where R′ is, e.g., H, alkyl, alkenyl, alkynyl, cycloalkyl, heterocyclyl, aryl, or heteroaryl, each of which is optionally substituted, exemplified by:

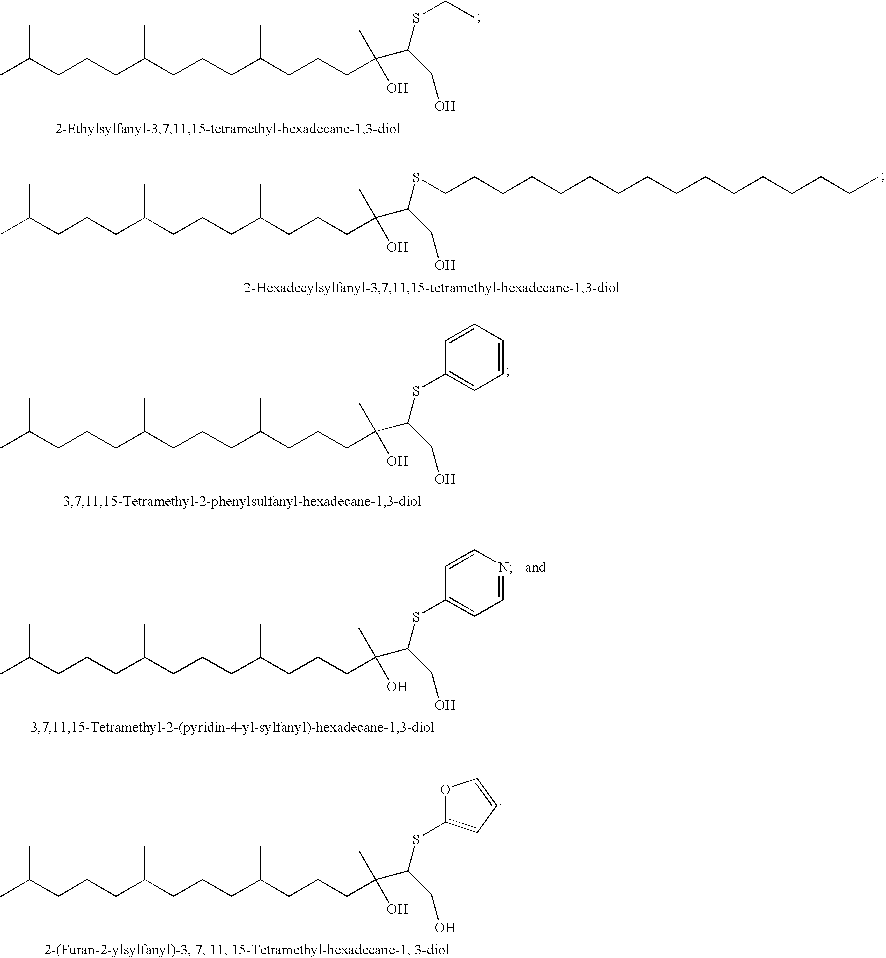

Compound class 36 are compounds with the general formula:

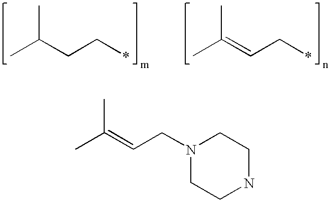

where n+m=an integer from 0 to 9, exemplified by:

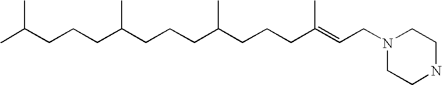

1-(3,7,11,15-Tetramethyl-hexadec-2-enyl)-piperazine.

Compound class 37 are compounds with the general formula:

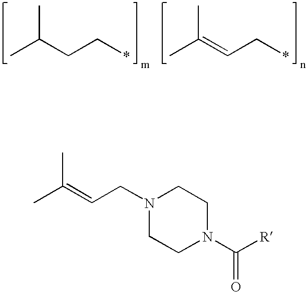

where n+m=an integer from 0 to 9; and where R′ is, e.g., H, alkyl, alkenyl, alkynyl, cycloalkyl, heterocyclyl, aryl, or heteroaryl, each of which is optionally substituted, exemplified by:

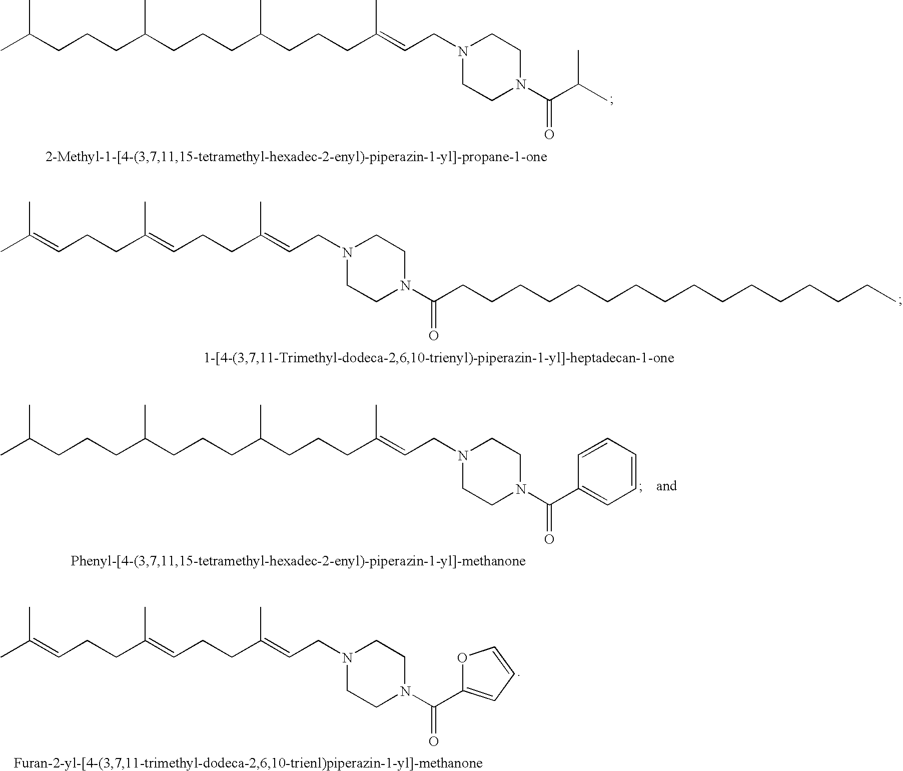

Compound class 38 are compounds with the general formula:

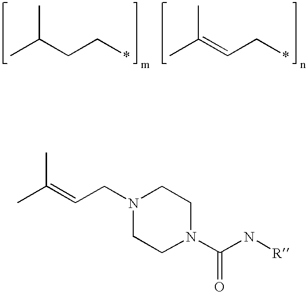

where n+m=an integer from 0 to 9; and where R″ is, e.g., H, alkyl, alkenyl, alkynyl, cycloalkyl, heterocyclyl, aryl, or heteroaryl, each of which is optionally substituted, exemplified by:

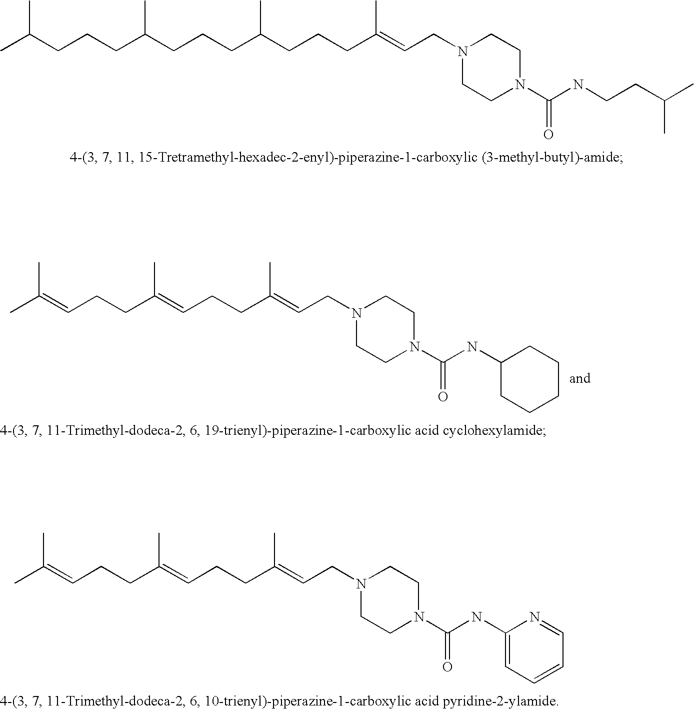

Compound class 39 are compounds with the general formula:

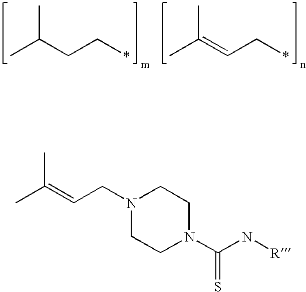

where n+m=an integer from 0 to 9; and where R′″ is, e.g., H, alkyl, alkenyl, alkynyl, cycloalkyl, heterocyclyl, aryl, or heteroaryl, each of which is optionally substituted, exemplified by:

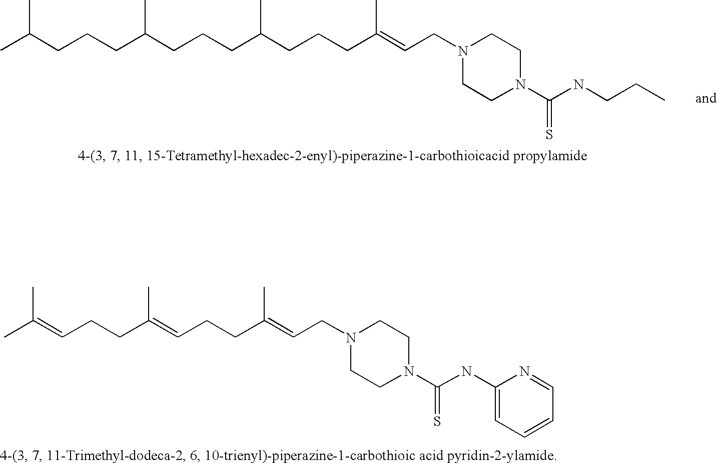

Compound 40 includes compounds with the general formula:

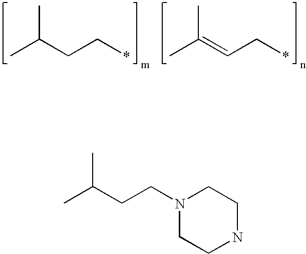

where n+m=an integer from 0 to 9; exemplified by:

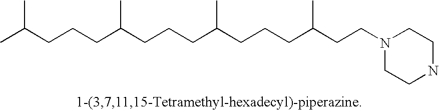

Compound class 41 are compounds having the general formula:

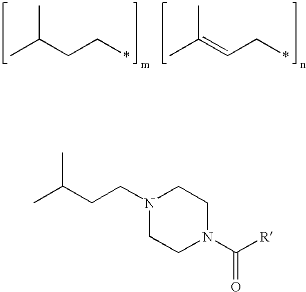

where n+m=an integer from 0 to 9; and where R′ is, e.g., H, alkyl, alkenyl, alkynyl, cycloalkyl, heterocyclyl, aryl, or heteroaryl, each of which is optionally substituted, exemplified by:

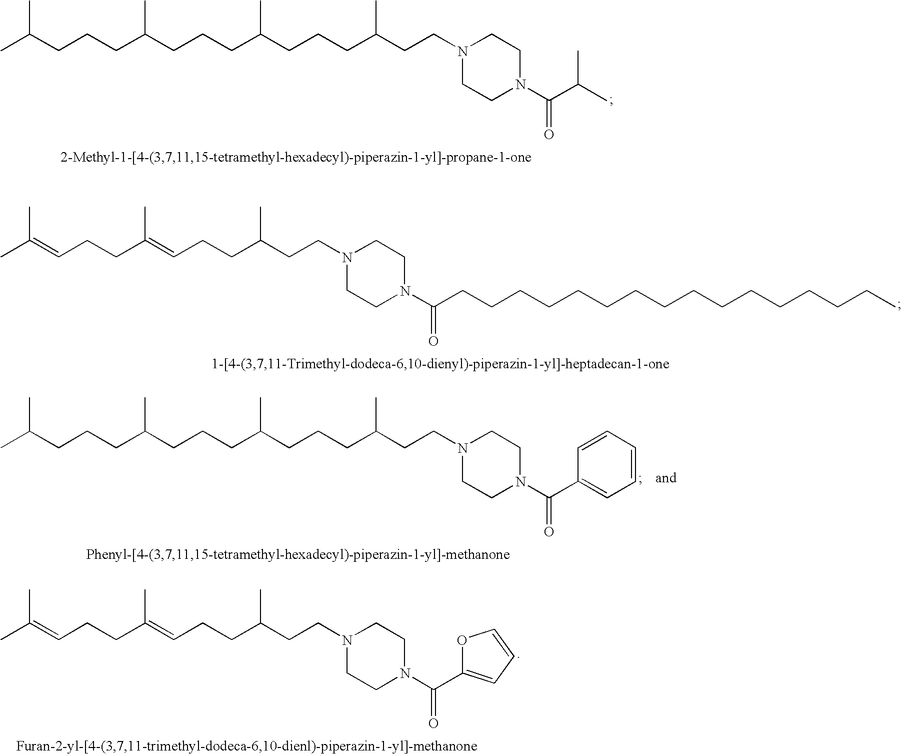

Compound class 42 are compounds with the general formula:

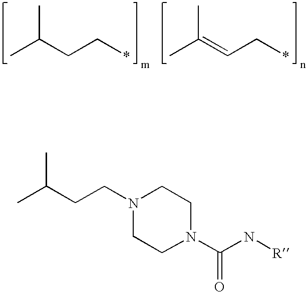

where n+m=an integer from 0 to 9; and where R″ is, e.g., H, alkyl, alkenyl, alkynyl, cycloalkyl, heterocyclyl, aryl, or heteroaryl, each of which is optionally substituted, exemplified by:

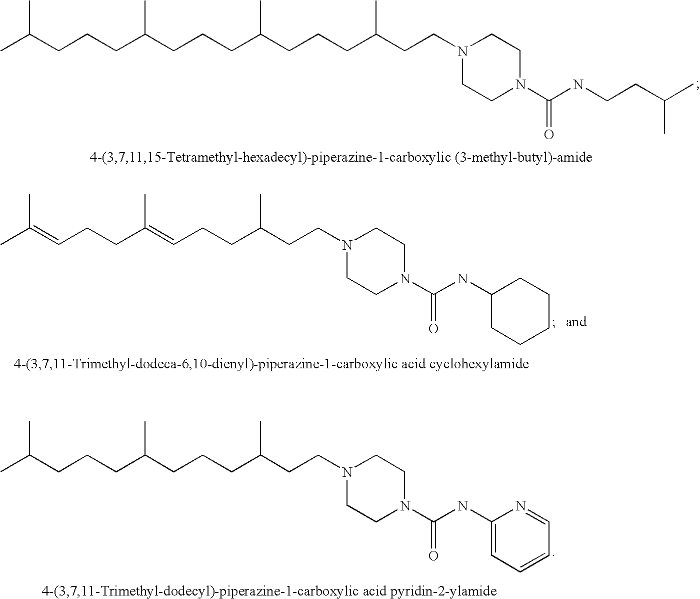

Compound class 43 are compounds with the general formula:

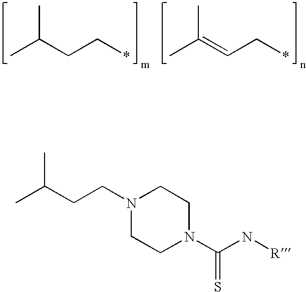

where n+m=an integer from 0 to 9; and where R′″ is, e.g., H, alkyl, alkenyl, alkynyl, cycloalkyl, heterocyclyl, aryl, or heteroaryl, each of which is optionally substituted, exemplified by:

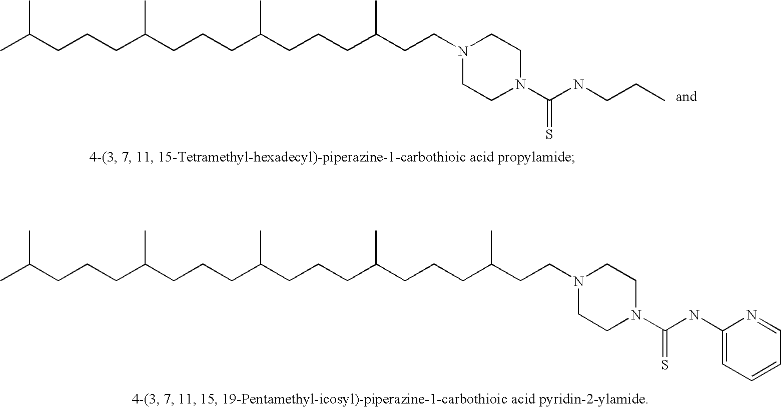

Compound class 44 includes ketones and aldehydes having the general formulas:

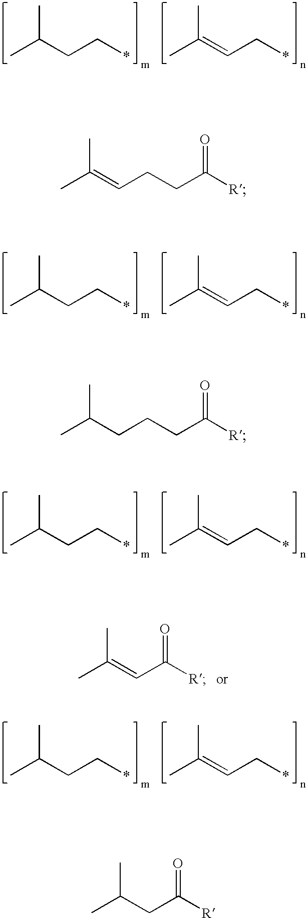

where n+m is an integer from 0 to 9; and where R′ can be, e.g., H, alkyl, alkenyl, alkynyl, cycloalkyl, heterocyclyl, aryl, or heteroaryl, each of which is optionally substituted. Particular compounds include:

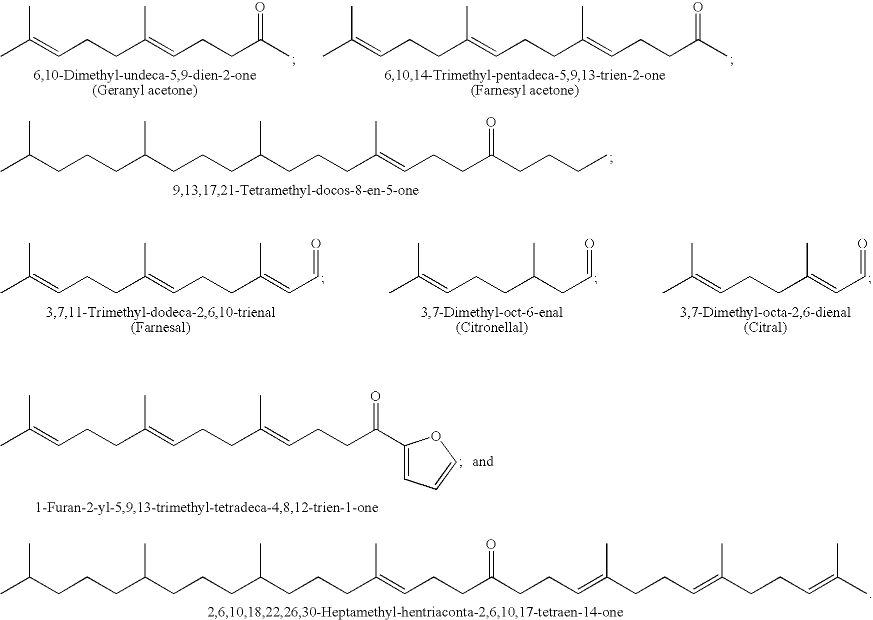

Compound class 45 are compounds having the general formula:

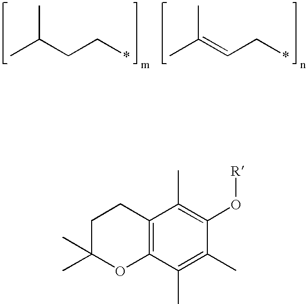

where n+m=an integer from 0 to 9; and where R′ can be, e.g., H; alkyl, alkenyl, alkynyl, cycloalkyl, each of which is optionally substituted (e.g., with halogen); exemplified by:

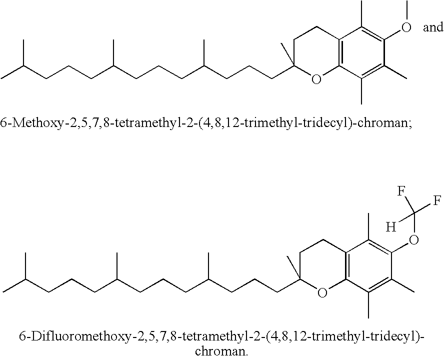

Compound Class 46 are compounds having the general formula:

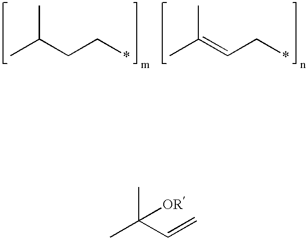

where n+m=an integer from 0 to 9; and where R′ can be H, alkyl, alkenyl, alkynyl, cycloalkyl, heterocyclyl, aryl, or heteroaryl, each of which is optionally substituted, exemplified by:

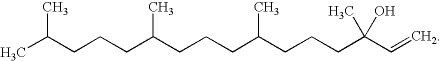

It is to be understood that any of the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

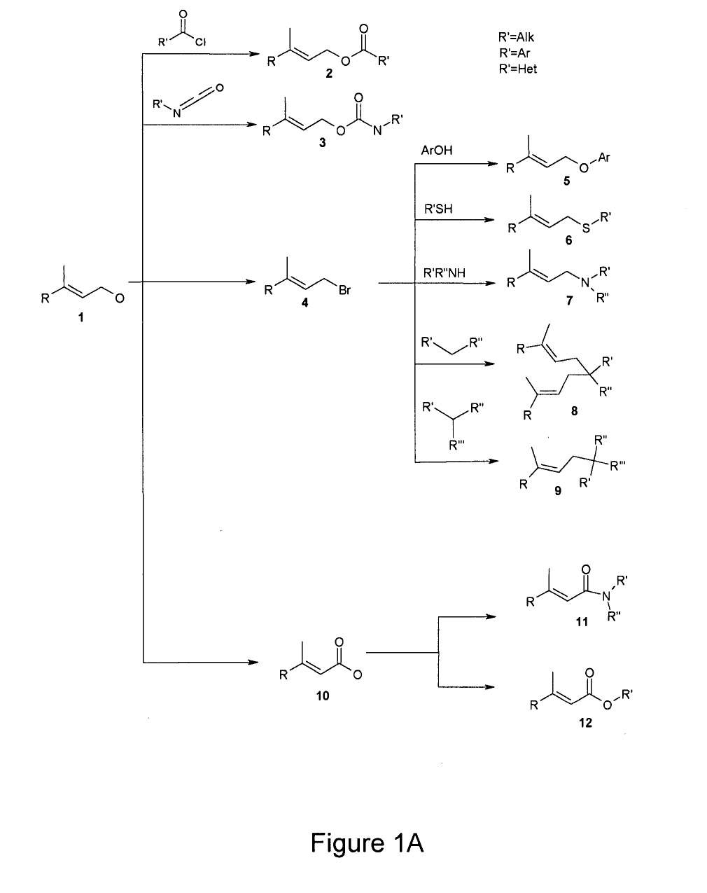

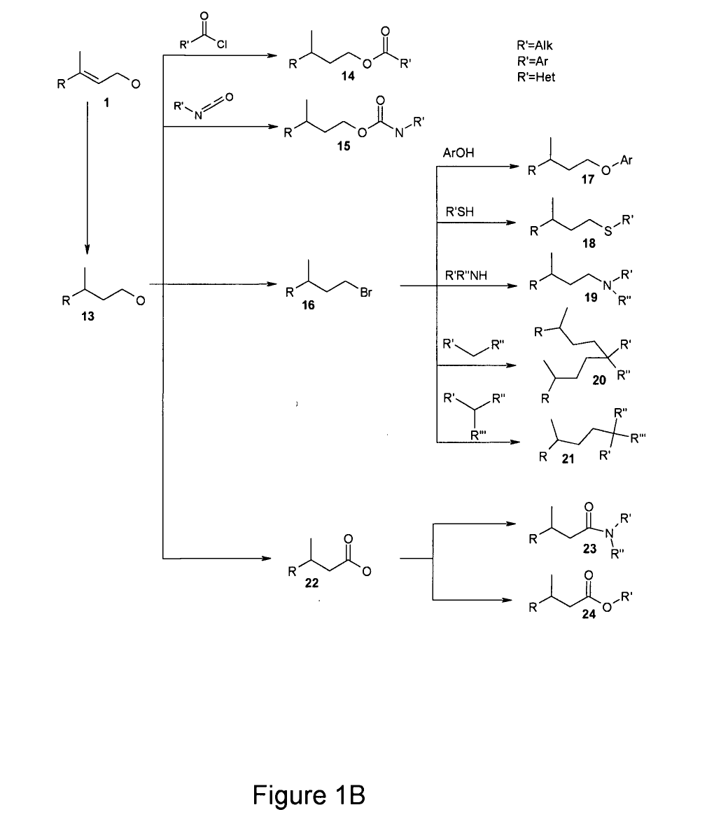

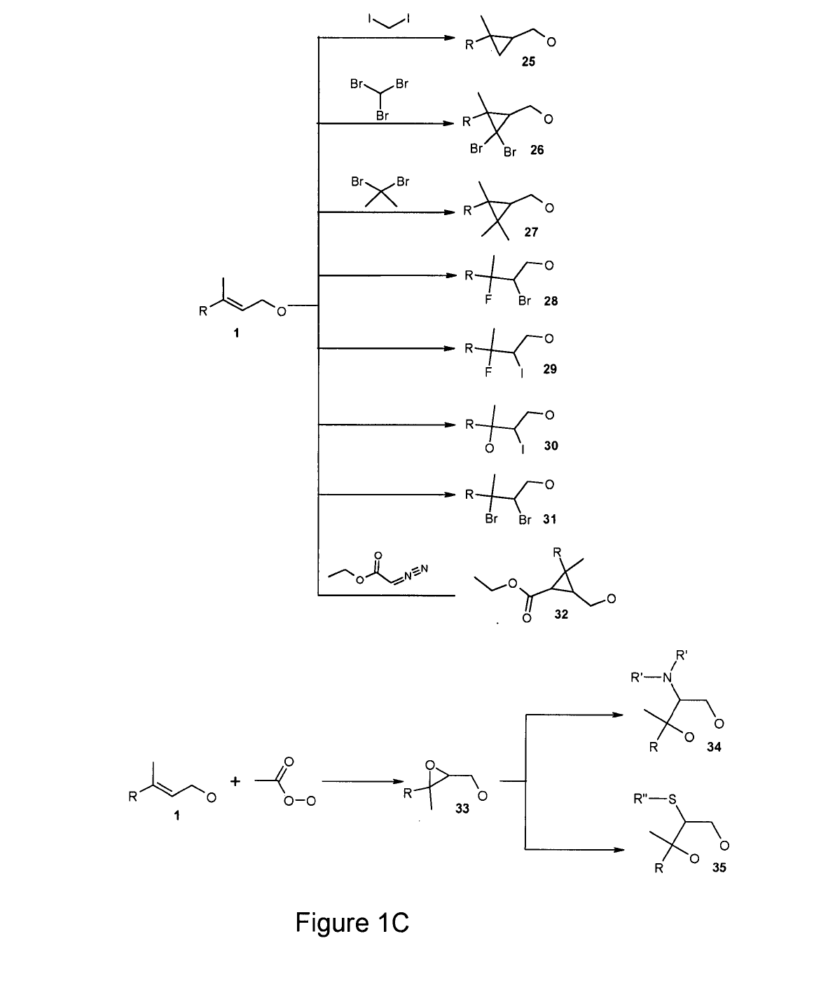

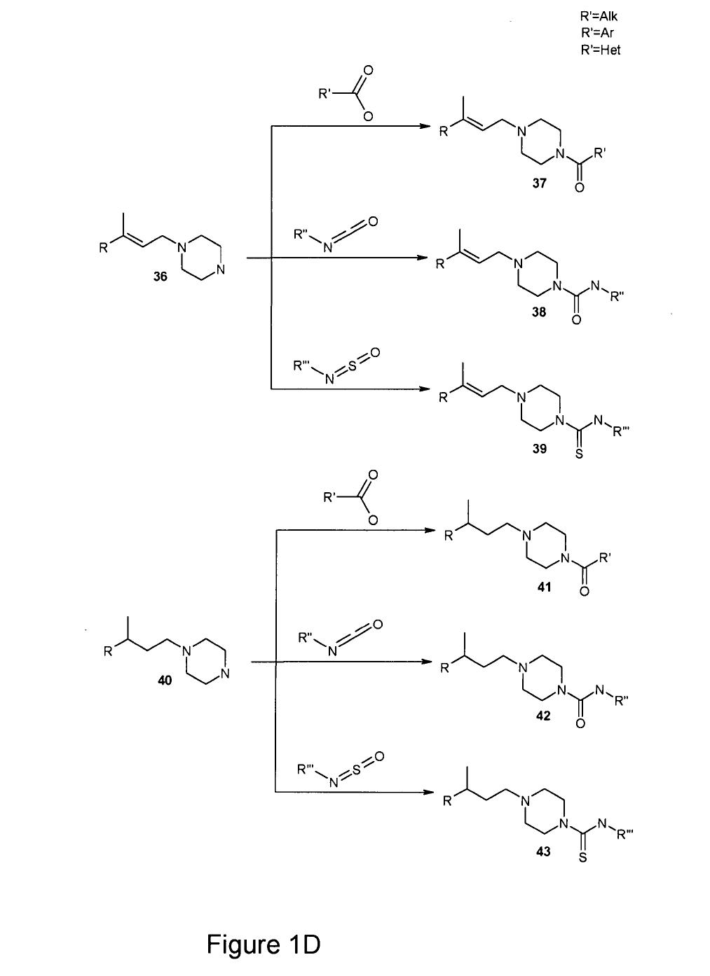

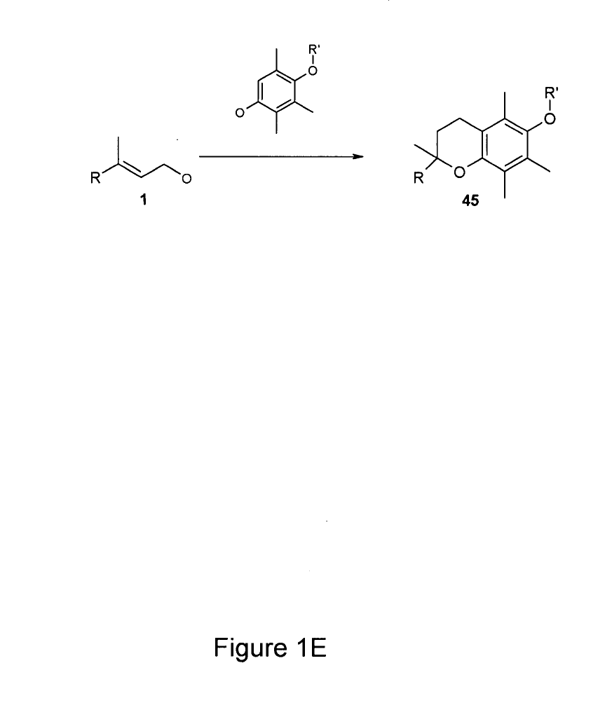

Compounds of the invention can be prepared by the methods set forth in FIG. 1A-1E. Starting materials, including isoprenoids, isoprenoid alcohols, polyprenols, terpenes, terpenoids, and terpenols, are available commercially from, e.g., Sigma-Aldrich (St. Louis, Mo.).

2. Treating, Preventing, Ameliorating Symptoms of or Delaying the Onset of an Autoimmune Condition

The invention provides methods, compounds, and compositions containing the compounds for treating, preventing, ameliorating one or more symptoms of, or delaying the onset of an autoimmune condition in a mammal. Methods for treating, preventing, delaying the onset of, or ameliorating one or more symptoms of an autoimmune condition include administering a compound that increases the level of NADPH oxidase activity in the mammal, e.g., an NADPH oxidase activator. For example, a compound that increases a cell's production of reactive oxygen species can be administered to a mammal with arthritis.

The invention further provides methods for the use of a compound that increases the level of NADPH oxidase activity in a mammal for the manufacture of a medicament for treating, preventing, delaying the onset of, or ameliorating one or more symptoms of an autoimmune condition.

Such autoimmune conditions include, without limitation, arthritis (e.g., rheumatoid arthritis (RA)), multiple sclerosis (MS), inflammatory bowel disease, Crohn disease, lupus, autoimmune uveitis, type I diabetes, bronchial asthma, septic arthritis induced with staphylococci or streptococci, and cardiovascular disease involving vasculitis.

Such compounds that increase NADPH oxidase activity can include any of the compounds described in the compound classes set forth above.

The compounds described herein can be administered in any standard form using any standard method (e.g., in the form of a composition, e.g., a pharmaceutical composition). For example, compounds that increase NADPH oxidase activity can be in the form of tablets or capsules (e.g., time-release capsules) that are taken orally. Alternatively, the compounds can be in a liquid form and can be taken orally or by injection. The compounds also can be in the form of suppositories. Further, compounds that increase NADPH oxidase activity can be in the form of creams, gels, and foams that can be applied to the skin. In addition, the compounds can in the form of an inhalant that is applied nasally. The compounds may be administered intra-dermally, intra-peritoneally, orally, or intra-nasally.

Compounds that increase NADPH oxidase activity can be administered at any dose that is sufficient to increase NADPH oxidase activity in cells that have low activity. Such doses can be taken over a period of years to prevent and/or delay the progression of the autoimmune condition or to reverse the progression of the autoimmune condition. Doses can be selected based on the effectiveness and toxicity of the particular compound using standard pharmacology techniques.

The methods of the invention can be applied to a wide range of subjects, e.g., mammals such as humans, non-human primates (e.g., monkeys), horses, cattle, pigs, sheep, goats, dogs, cats, rabbits, guinea pigs, hamsters, rats, and mice. In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In one in vivo approach, a compound or pharmaceutical composition described herein is administered to the subject, e.g., a mammal, such as a mammal suspected of suffering from, or suffering from, an autoimmune condition. Generally, the compounds of the invention will be suspended in a pharmaceutically-acceptable carrier (e.g., physiological saline) and administered orally or transdermally or injected (or infused) intravenously, subcutaneously, intramuscularly, intraperitoneally, intrarectally, intravaginally, intranasally, intragastrically, intratracheally, or intrapulmonarily. They can be delivered directly to an appropriate affected tissue.

The dosages of the inhibitory compounds and supplementary agents to be used depend on the choice of the route of administration; the nature of the formulation; the nature of the patient's illness; the subject's size, weight, surface area, age, and sex; other drugs being administered; and the judgment of the attending physician. Suitable dosages are generally in the range of 0.0001-100.0 mg/kg. Wide variations in the needed dosage are to be expected in view of the variety of compounds and supplementary agents available and the differing efficiencies of various routes of administration. For example, oral administration would be expected to require higher dosages than administration by i.v. injection. Variations in these dosage levels can be adjusted using standard empirical routines for optimization as is well understood in the art. Administrations of compounds and/or supplementary agents can be single or multiple (e.g., 2-, 3-, 4-, 6-, 8-, 10-, 20-, 50-,100-, 150-, or more fold).

The invention will be further described in the following examples, which do not limit the scope of the invention described in the claims.

### EXAMPLES

### Example 1

Animals

Rat (Rattus norvegicus) strains used in the following experiments included the DA and LEW.1 F strains, which are highly susceptible to PIA and EAE, and the E3 strain, which is PIA and EAE-resistant (Bergsteinsdottir et al. (2000) J Immunol 164: 1564-8; Vingsbo et al. (1996) Am J Pathol 149: 1675-83). DA, LEW.1F and E3 rats were obtained from Zentralinstitut für Versuchstierzucht, Hannover, Germany; DA rats purchased from Harlan Netherlands were also used.

The term DA.Ncf1^{DA} rats designates DA rats carrying the Ncf1 DA allele with impaired NADPH oxidase capacity and the term DA.Ncf1^{E3} rats designates DA rats carrying the Ncf1^{E3} allele with functional NADPH oxidase capacity (Olofsson & Holmdahl (2003) Scand J Immunol 58:155-164).

All animals were kept in animal facilities that have climate-controlled environments with 12-hour light/dark cycles. Rats were housed in polystyrene cages containing wood shavings and were fed standard rodent chow and water ad libitum. Rats were free from common pathogens, including the Sendai virus, Hantaan virus, coronavirus, reovirus, cytomegalovirus, and Mycoplasma pulmonis.

Disease was induced in all rats at the age of 6-12 weeks. Arthritis development was monitored with a macroscopic scoring system of the four limbs ranging from 0 to 15 (1 point for each swollen or red toe, 1 point for midfoot digit or knuckle, 5 points for a swollen ankle). The scores of the four paws were added yielding a maximum total score of 60 for each rat.

### Example 2

Compounds
Compound 1: Phytol (3,7,11,15-Tetramethyl-2-hexadecen-1-ol) 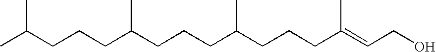
was obtained from Sigma Aldrich.
Compound 2: Farnesol (3,7,11-Trimethyl-2,6,10-dodecatrien-1-ol) 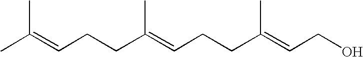
was obtained from Sigma Aldrich.
Compound 3: Geranylgeraniol (3,7,11,15-Tetramethyl-hexada-2,6,10,14-tetraen-1-ol) 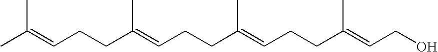
was obtained from Sigma Aldrich.
Compound 4: Farnesyl acetate (Acetic acid 3,7,11-trimethyl-dodeca-2,6,10-trienyl ester) 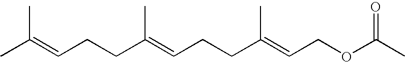
was obtained from Fluka.
Compound 5: Phytyl acetate (Acetic acid 3,7,11,15-tetra-metyl-hexadec-2-enyl-ester) 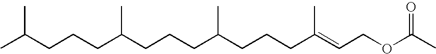
was synthesized according to FIG. 1A.
Compound 6: Di-hydro-phytol (3,7,11,15-Tetramethyl-hexadecan-1-ol) 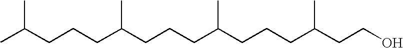
was synthesized according to FIG. 1B.
Compound 7: Di-hydro-phytyl acetate (Acetic acid 3,7,11,15-tetramethyl-hexadecyl ester) 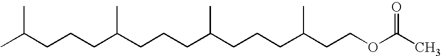
was synthesized according to FIG. 1B.
Compound 8: 6-Difluoromethoxy-2,5,7,8-tetramethyl-2-(4,8, 12-trimethyl-tridecyl)-chroman 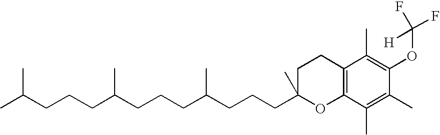
was synthesized according to FIG. 1E.
Compound 9: Isophytol (3,7,11,15-Tetramethyl-1-hexadecen-3-ol) 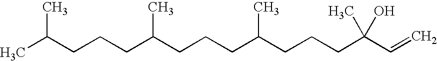
was obtained from Sigma Aldrich.

### Example 3

Oxidative Burst Assay of Granulocytes In Vitro

The human myeloma cell line HL-60 (ATCC, CCI-240) was cultured in D-MEM (Gibco, Paisley, UK) complemented with 10% fetal calf serum and Penicillin-Streptomycin. The cells were differentiated to granulocytes by culture in presence of 1.25% dimethylsulfoxide (DMSO; Sigma Aldrich Inc., Milwaukee, Wis.) for six days. Before assaying the cells were washed and resuspended in PBS to a concentration of 10⁷ cells/ml.

Oxidative Burst Assay of Granulocytes In Vitro

NADPH activating oils were tested for oxidative burst inducing capacity according to a previously described method (Dahlgren and Karlsson (1999) J Immunol Methods 232:3-14). In order to get the oils solubilized, they were diluted at 2% concentration in β-cyclodextrin (Sigma Aldrich) (10 mM in PBS) in PBS. β-cyclodextrin by itself or the used oil control had no stimulating effect on ROS production. Briefly, 5 μl of resuspended oils were added into 96-well plates containing 5×10⁵ cells/well in a total volume of 200 μl PBS/well (final concentration isoluminol 50 μg/ml; Sigma Aldrich) and horse radish peroxidase (type II 2,5 U/ml; Sigma Aldrich). Samples were gently mixed and the data collection was initiated immediately. Extra-cellular ROS production was followed at 37° C. as produced luminescence signal (FluoStar Optima, BMG Labtechnologies, Offenburg, Germany) and presented as maximal relative signal during a measurement period of 50 minutes.

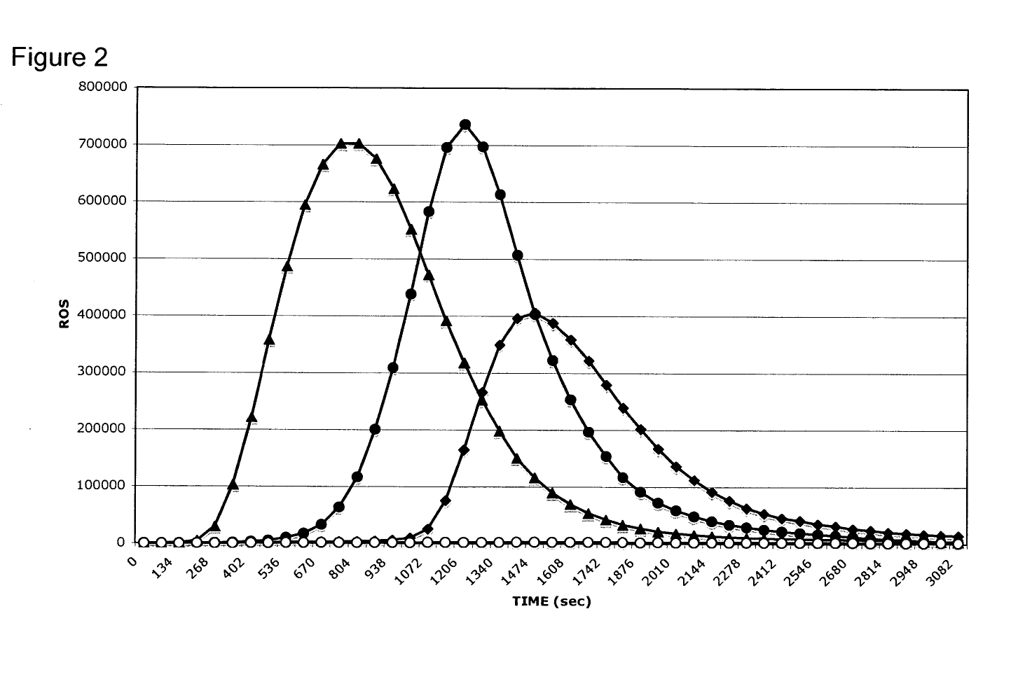

The NADPH activators compound 1 (phytol), compound 2 (farnesol) and compound 3 (geranylgeraniol) give strong ROS production from the HL60 cells. These compounds are all non arthritogenic but have arthritis ameliorating effects. Pristane is a potent arthritis inducing compound that does not induce ROS production in HL60 cells. Extra-cellular ROS production was measured with an isoluminol assay on HL-60 cells after in vitro stimulation with compound 1 (phytol) (♦), compound 2 (farnesol) (●), compound 3 (geranylgeraniol) (▴) or pristane (◯). (FIG. 2).

### Example 4

Compound 1 (Phytol) Increases Oxidative Burst In Vivo

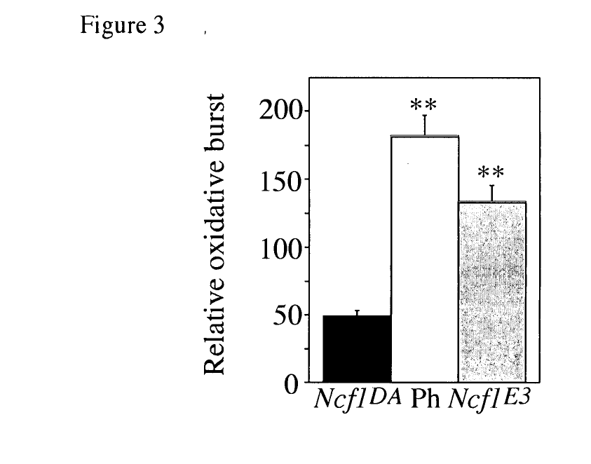

Phytol restores the oxidative burst defect in vivo. Oxidative burst response to PMA stimulation in vitro in spleen granulocytes taken 5 days after s.c. phytol injection in DA.Ncf1^{DA} rats (200 μl) compared to naive DA.Ncf1^{DA} and DA.Ncf1^{E3} (n=5) (FIG. 3).

Treatment of rats with impaired NADPH oxidase capacity, DA.Ncf1^{DA} rats, with injection of compound 1 restores to NADPH oxidase capacity to a level comparable to the level of NADPH oxidase capacity in rats with a functional oxidative burst, DA.Ncf1^{E3} rats.

### Example 5

Biodistribution

DA rats originating from Harlan Netherlands (N=4 rats per group) injected with compound 1 (phytol) mixed with radiolabelled compound 1 (phytol) (Moravek Biochemicals, CA) to a total volume of 2001 μl at 167 μCi/rat with ³H-Phytol. At sacrifice at day 2, 4, 8, 11 and 17, respectively, inguinal lymph nodes, spleen, heart, thymus, kidney, liver, lung, adipose tissue, muscle, injection site tissue and blood were collected in equal amount of saline solution in pre-weight tubes (blood with heparin to prevent coagulation). The tissues were homogenized and mixed with ready-safe scintillation liquid (Beckman Coulter, CA).

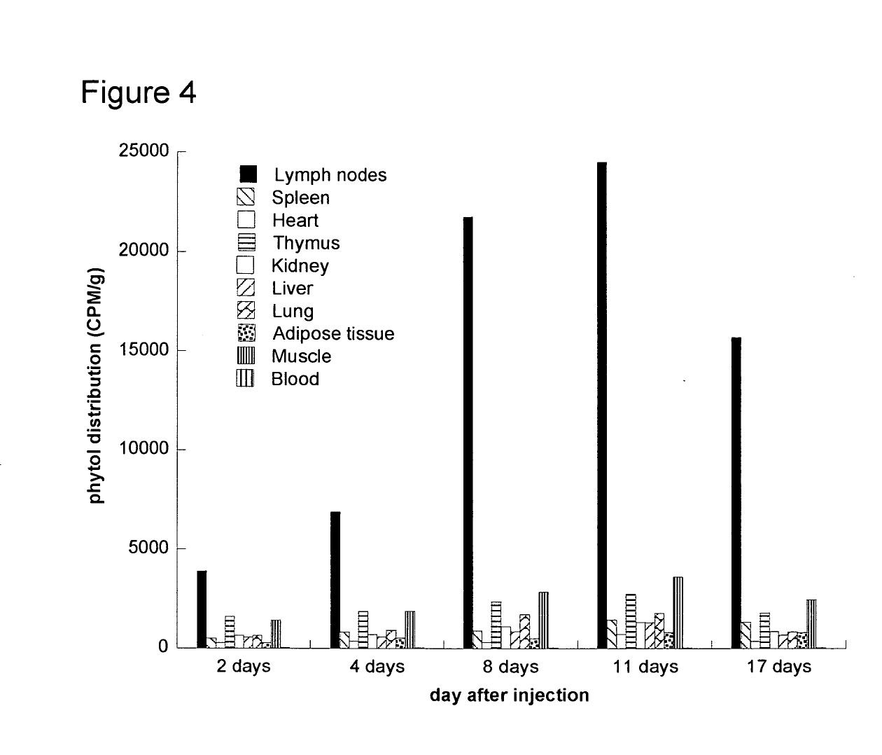

Biodistribution was estimated as relative CPM per gram tissue. Besides the large fraction of phytol (>90%, not shown) that remained as a depot in the injection site, the draining lymph nodes were the primary tissue for accumulation of phytol. The distribution of phytol to the inguinal lymph nodes showed highest accumulation more than one week after injection and showed a decrease after two weeks (FIG. 4). It was at this level determined that phytol was distributed primarily to the cortical regions as well as close to the sinusoidal space of the draining lymph nodes. Moreover, the staining seemed to appear in-between cells or in the cell membrane, i.e. not intracellular.

### Example 6

Treatment of PIA With Compound 1 (Phytol)

Pristane induced arthritis (PIA) was induced in the rats by a subcutaneous (s.c.) injection at the base of the tail with 200 μl of pristane.

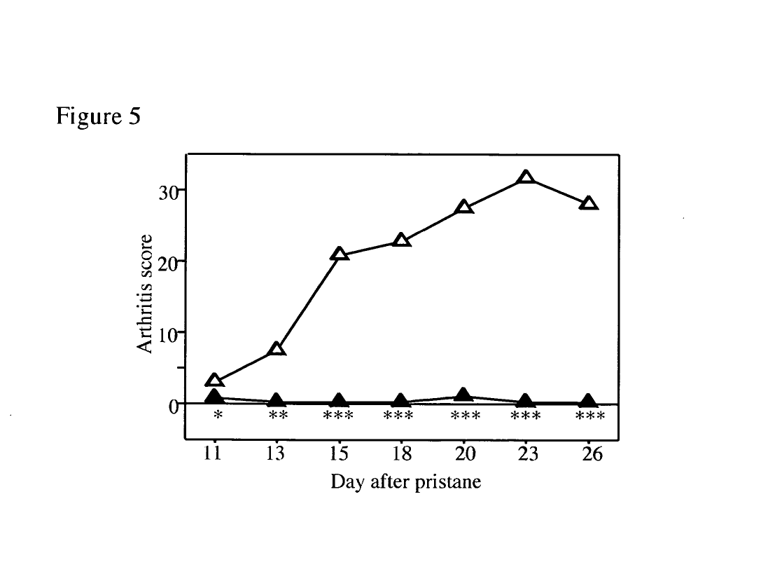

Compound 1 (phytol) inhibits development of arthritis. A significant preventive effect of compound 1 (phytol) on PIA can be seen in DA.Ncf1^{DA} rats. Compound 1 (phytol) was injected s.c. day-5 prior to pristane injection (untreated control (Δ) and compound 1 (phytol) (▴)). (FIG. 5).

### Example 7

Treatment of Established PIA With Compound 1 (Phytol)

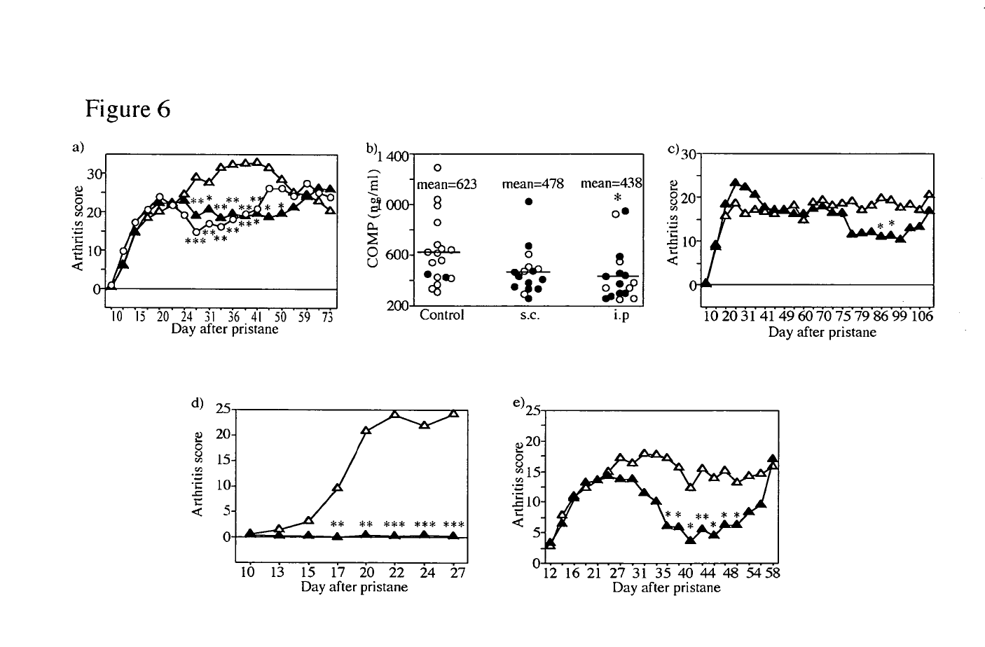

Pristane induced arthritis (PIA) was induced in the rats by an subcutaneous (s.c.) injection at the base of the tail with 200 μl of pristane. Therapeutic treatments were administered s.c. or intra peritoneal (i.p.; 200 μl) after onset of disease in the acute or chronic phase (FIG. 6).

Injection of compound 1 (phytol) ameliorates disease in the acute phase of arthritis (FIG. 6A). A significant therapeutic effect of compound 1 (phytol) can be seen in acute phase of PIA in DA.Ncf1^{DA} rats after two injections (200 μl) with 5 days in between starting at the estimated peak of disease (day 22).

Cartilage destruction is decreased after injection of compound 1 (phytol) (FIG. 6B). Sera were taken day 38 and analyzed for COMP levels as a measurement of cartilage destruction. Results are presented as circles, where every circle represents one individual and filled circles represent rats with a mean reduction in score after day of first treatment (day 22). Lines represent mean value.

Compound 1 (phytol) decreases arthritis severity in the chronic phase of arthritis (FIG. 6C). Compound 1 (phytol) was injected i.p. twice (200 μl), starting day 70 after pristane injection in DA.Ncf1^{DA} rats with three days between treatments (control (Δ) and compound 1 (phytol) s.c. (▴)).

The preventive effect of compound 1 (phytol) is not dependent on genetic background (FIG. 6D). An arthritis preventing effect of compound 1 (phytol) when injected (200 μl s.c.) 5 days before induction of PIA could also be seen in another arthritis susceptible strain i.e. the Lewis.IF rat, (control (Δ) and compound 1 (phytol) s.c. (▴)).

Compound 1 (phytol) has an arthritis ameliorating effect also in rats with a functional oxidative burst (FIG. 6E). Rats heterozygous for the functional Ncf1 (Ncf1^{E3}) allele but with an increased arthritis susceptibility locus (Pia3) introduced in the genome on chromosome 6 (Olofsson et al. (2003) Genomics 82: 652-9). Compound 1 (phytol) was injected i.p. (200 μl) at the estimated peak of disease (control (Δ) and compound 1 (phytol) (▴)).

### Example 8

Compound 1 (Phytol) Suppresses CIA With Oil Adjuvant

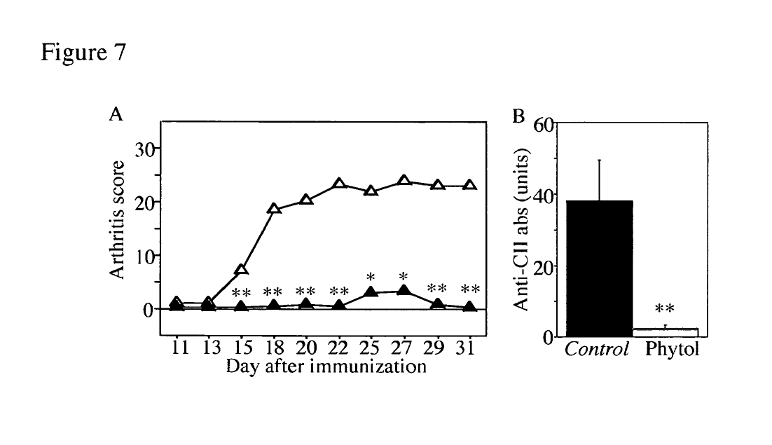

Collagen induced arthritis (CIA) was induced by an s.c. injection of 100 μl of rat collagen type II (CII; 100 μg/rat-150 μg/rat), emulsified in incomplete Freund's adjuvant (IFA; Sigma Aldrich) (FIG. 7).

Compound 1 (phytol) prevents collagen-induced arthritis in DA.Ncf1^{DA} rats (FIG. 7A). Effect of s.c. administration of phytol (200 μl) 5 days.

Compound 1 (phytol) decreases anti-CII antibody levels in serum (FIG. 7B). At day 27 after immunization, sera were taken and analyzed for anti-CII IgG levels (n=7). Titers are relative to pooled positive sera (units).

### Example 9

Compound 1 (Phytol) Suppresses CIA Without Oil Adjuvant

Non oil collagen induced arthritis (NOCIA) was induced by emulsifying rat CII (300 μg) in a mixture of LPS (50 μg; Sigma Aldrich), CPG (5′-TCC ATG ACG TTC CTG ACG TT-3′; SEQ ID NO:1) (45 μg; MWG-Biotech AG, Ebersberg, Germany) and alum (6 mg; Sigma Aldrich) and injected s.c. in a total volume of 300 μl.

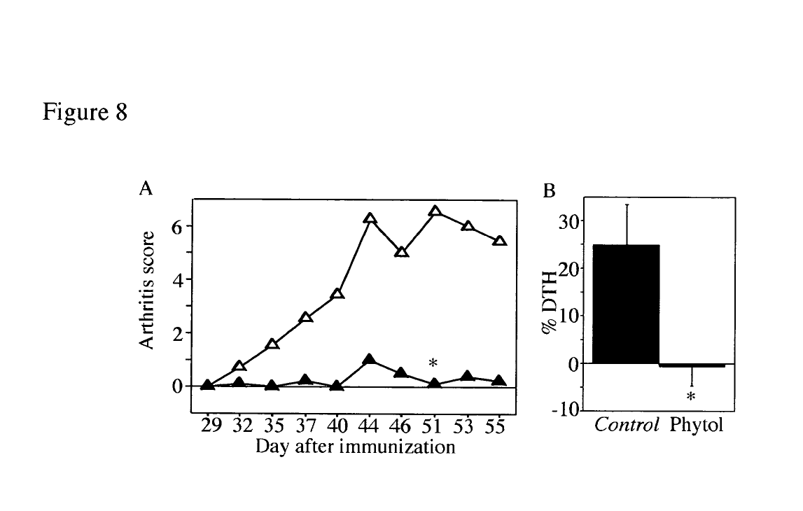

Compound 1 (phytol) reduces arthritis severity also in NOCIA (FIG. 8A), an arthritis model induced without involvement of oil. Preventive effect on arthritis of phytol injection (200 μl s.c.) five days prior to induction of NOCIA in DA.Ncf1^{DA} rats, (control (Δ) and compound 1 (phytol) (▴)).

Delayed type hypersensitivity (DTH) response is abrogated after compound 1 (phytol) injection (FIG. 8B). Day 67 after NOCIA immunization the immune system was challenged by an injection of CII in the ear. Following increase in ear volume was measured and compared to control ear (control n=7 and compound 1 (phytol) n=8).

### Example 10

Arthritis Treatment With Compound 5 (Phytyl Acetate)

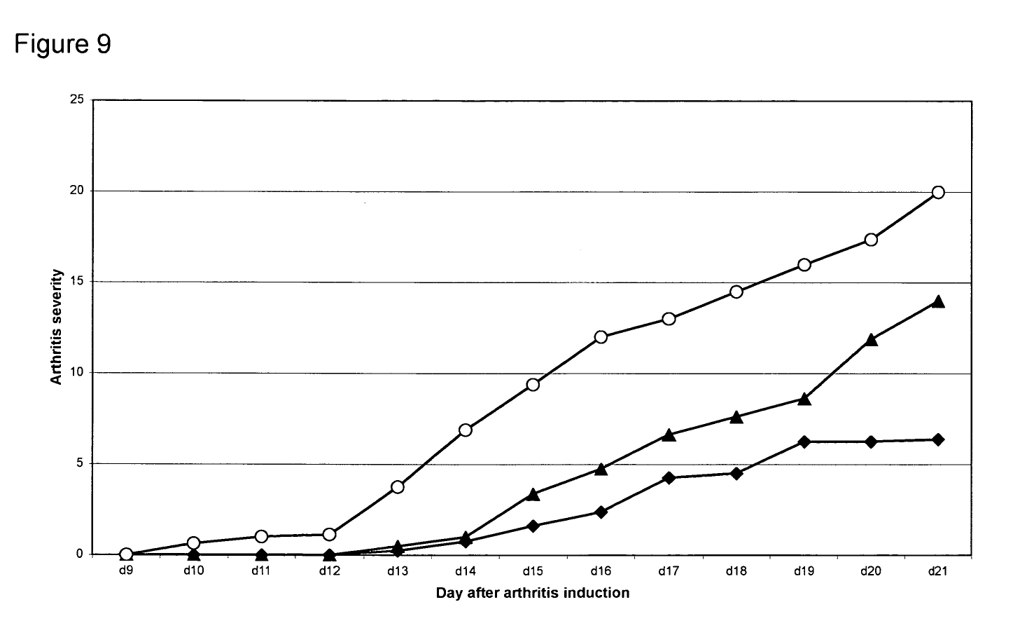

Pristane induced arthritis (PIA) was induced in the rats by a subcutaneous (s.c.) injection at the base of the tail with 150 μl of pristane. Treatments with compound 1 (phytol) or compound 5 (phytyl acetate) were administered to DA.Ncf1^{DA} rats as 100 μl subcutaneous injections in the neck at day 8 and 10 after arthritis induction (untreated control (◯), compound 1 (phytol) (♦) and compound 5 (phytyl acetate) (▴)) (FIG. 9).

Treatment with compound 5, which is derivatized with a functional moiety, a carboxylic acid ester, gives a therapeutic effect comparable to the therapeutic effect of the un-derivatized compound 1.

### Example 11

Arthritis Treatment With Compound 8

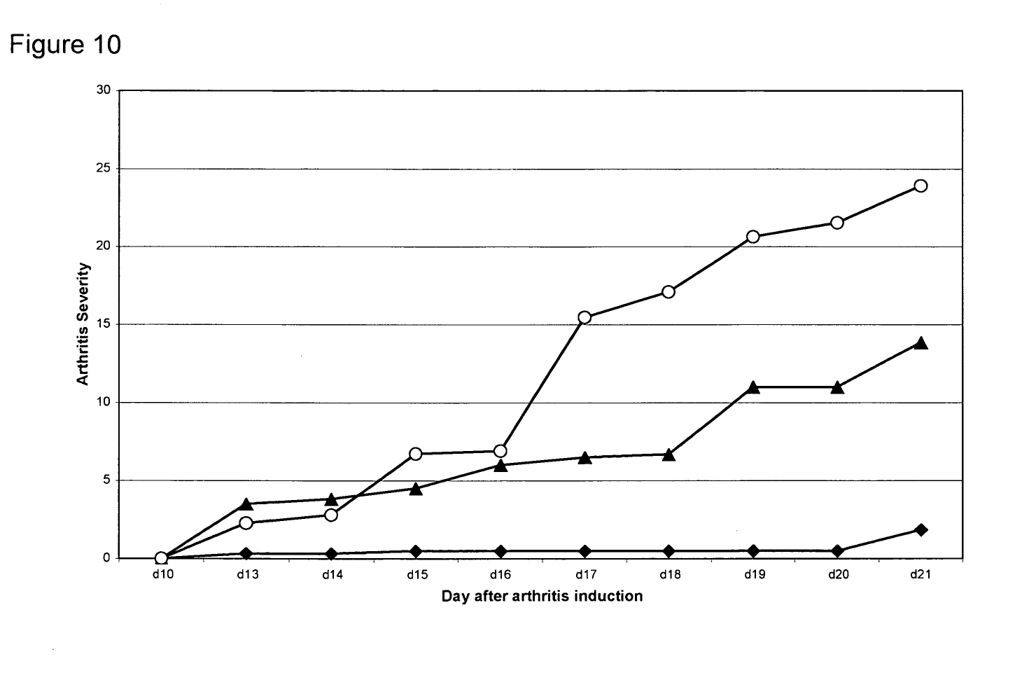

Pristane induced arthritis (PIA) was induced in the rats by a subcutaneous (s.c.) injection at the base of the tail with 150 μl of pristane. Treatments with compound 8 or compound 1 (phytol) were administered to DA.Ncf1^{DA} rats as 100 μl subcutaneous injections in the neck at day 8 and 10 after arthritis induction, (untreated control (◯), compound 1 (phytol) (♦) and compound 8 (▴)) (FIG. 10).

### Example 12

Arthritis Treatment With Compound 2 (Farnesol) and Compound 4 (Farnesylacetate)

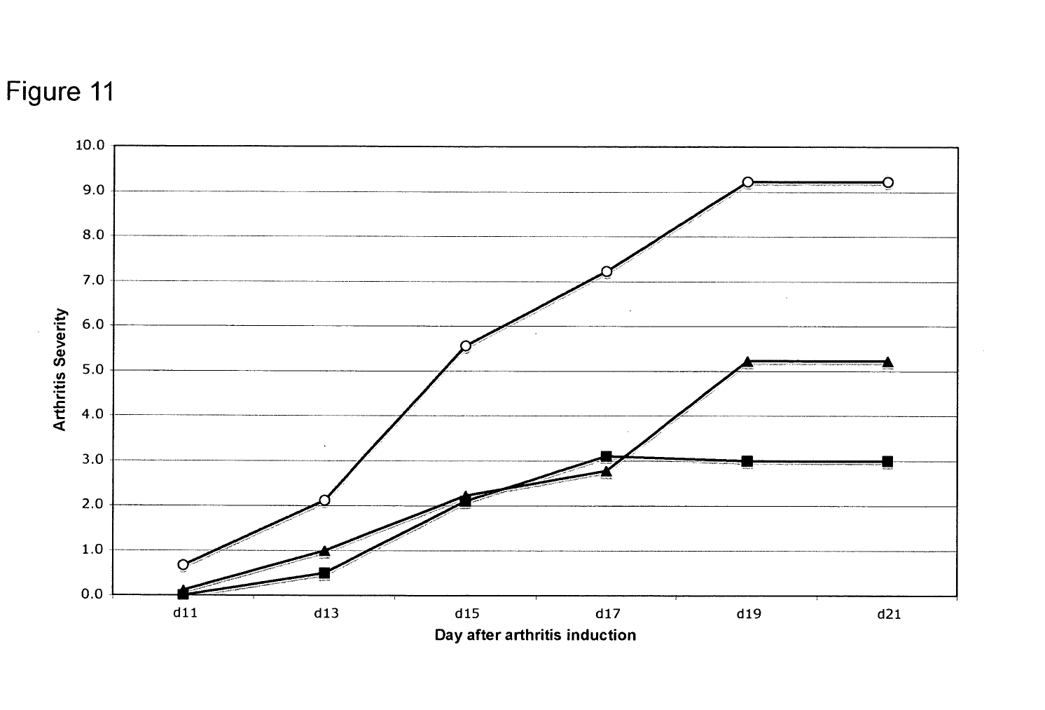

Pristane induced arthritis (PIA) was induced in the rats by a subcutaneous (s.c.) injection at the base of the tail with 150 μl of pristane. Treatments with compound 2 (farnesol) or compound 4 (farnesylacetate) were administered to DA.Ncf1^{DA} rats as 100 μl subcutaneous injections in the neck at day 8 and 10 after arthritis induction, (untreated control (◯), compound 2 (farnesol) (▪) and compound 4 (farnesylacetate) (▴)) (FIG. 11).

Treatment with compound 4, which is derivatized with a functional moiety, a carboxylic acid ester, gives a therapeutic effect comparable to the therapeutic effect of the un-derivatized compound 2.

### Example 13

Arthritis Treatment With Compound 1 (Phytol), Compound 6 (Di-Hydro-Phytol, Compound 5 (Phytyl Acetate), and Compound 7 (Di-Hydro-Phytyl Acetate

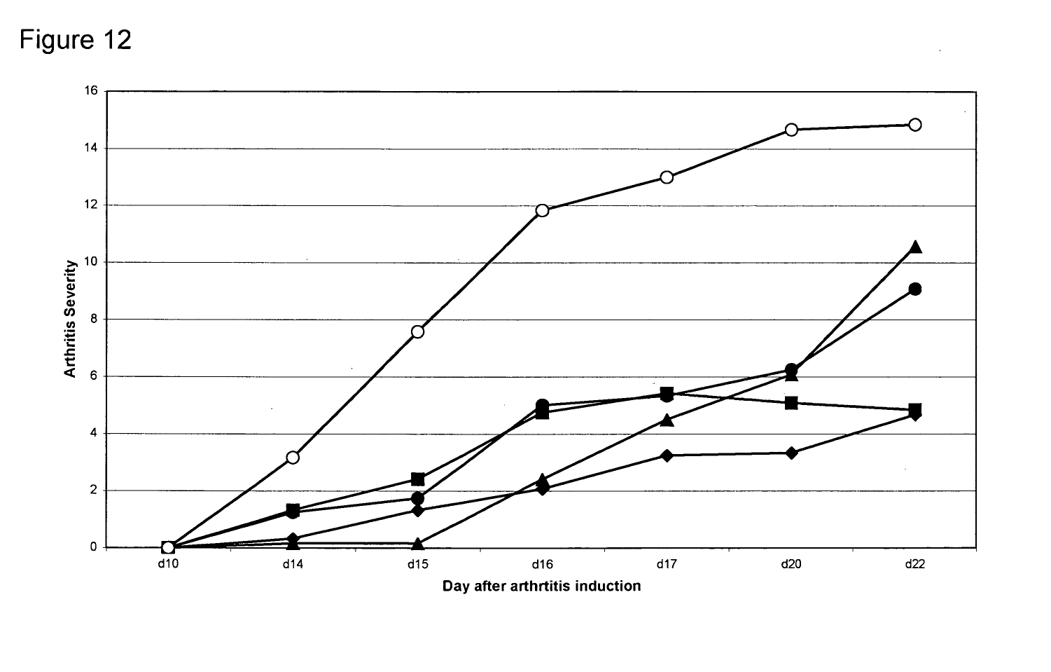

Pristane induced arthritis (PIA) was induced in the rats by a subcutaneous (s.c.) injection at the base of the tail with 150 μl of pristane. Treatments with compound 1 (phytol), compound 6 (di-hydro-phytol, compound 5 (phytyl acetate), or compound 7 (di-hydro-phytyl acetate were administered to DA.Ncf1^{DA} rats as 100 μl subcutaneous injections in the neck at day 8 and 10 after arthritis induction, (untreated control (◯), compound 1 (phytol) (♦), compound 6 (di-hydro-phytol (▪), compound 5 (phytyl acetate) (▴) or compound 7 (di-hydro-phytyl acetate (●)) (FIG. 12).

Treatment with compound 6 and compound 7, which are compounds comprising saturated isoprenoid units, gives a therapeutic effect comparable to the therapeutic effects of compound 1 and compound 5 which comprise one unsaturated isoprenoid unit.

### Example 14

Arthritis Treatment With Oral Administration of Compound 1 (Phytol)

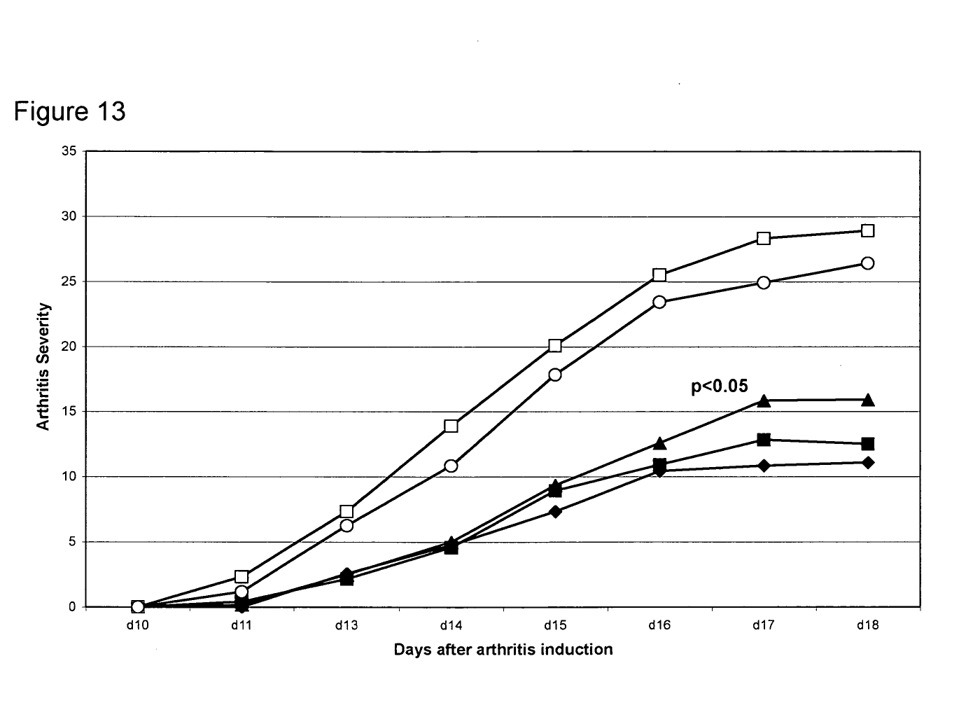

Pristane induced arthritis (PIA) was induced in the rats by an subcutaneous (s.c.) injection at the base of the tail with 150 μl of pristane. Treatments of DA.Ncf1^{DA} rats with compound 1 (phytol) mixed in the food for continuous uptake compared with Omega3 fatty acids, (untreated control (◯), Omega3 fatty acid (□), phytol 1% daily (♦), phytol 1% every 2'nd day (▴), or phytol 1% initiated day 7 after arthritis induction (▪)) (FIG. 13).

All three regimes of oral treatment with compound 1 (phytol) reduced the severity of arthritis in the animals. Treatment with omega3 fatty acids had no effect on the severity of arthritis.

### Example 15

Arthritis Treatment With Oral Administration of Compound 5 (Phytol Acetate)

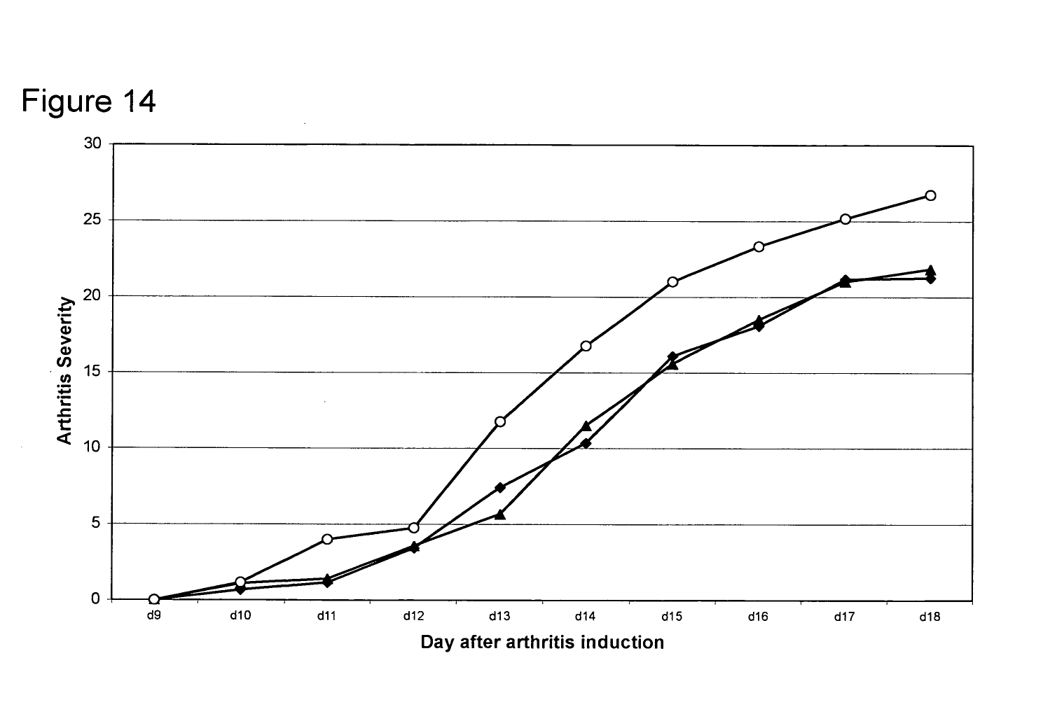

Pristane induced arthritis (PIA) was induced in the rats by an subcutaneous (s.c.) injection at the base of the tail with 150 μl of pristane. Treatments of DA.Ncf1^{DA} rats with compound 5 (phytyl acetate) mixed in the food for daily uptake, (untreated control (◯), compound 1 (phytol) 1% daily (♦), or compound 5 (phytyl acetate) 1% daily (▴)) (FIG. 14).

Oral treatment with compound 5 (phytyl acetate) was equally effective as oral treatment with compound 1 (phytol) in reducing the severity of arthritis.

### Example 16

Oxidative Burst Assay of Granulocytes In Vitro

The human myeloma cells line HL-60 (ATCC, CCI-240) was cultured in D-MEM (Gibco, Paisley, UK) complemented with 10% fetal calf serum and Penicillin-Streptomycin. The cells were differentiated to granulocytes by culture in presence of 1,25% dimethylsulfoxide (DMSO; Sigma Aldrich Inc., Milwaukee, Wis.) for six days. Before assaying the cells were washed and resuspended in PBS to a concentration of 10⁷ cells/ml.

Oxidative Burst Assay of Granulocytes In Vitro

NADPH activating oils were tested for oxidative burst inducing capacity according to a previously described method (Dahlgren and Karlsson (1999) J Immunol Methods 232: 3-14). In order to get the oils soluble they were diluted at 2% concentration in β-cyclodextrin (Sigma Aldrich) (10 mM in PBS) in PBS. β-cyclodextrin by itself or the used oil control had no stimulating effect on ROS production. Briefly, 5 μl of resuspended oils were added into 96-well plates containing 5×10⁵ cells/well in a total volume of 200 μl PBS/well (final concentration isoluminol 50 μg/ml; Sigma Aldrich) and horseradish peroxidase (type II 2,5 U/ml; Sigma Aldrich). Samples were gently mixed and the data collection was initiated immediately. Extra-cellular ROS production was followed at 37° C. as produced luminescence signal (FluoStar Optima, BMG Labtechnologies, Offenburg, Germany) and presented as maximal relative signal during a measurement period of 50 minutes.

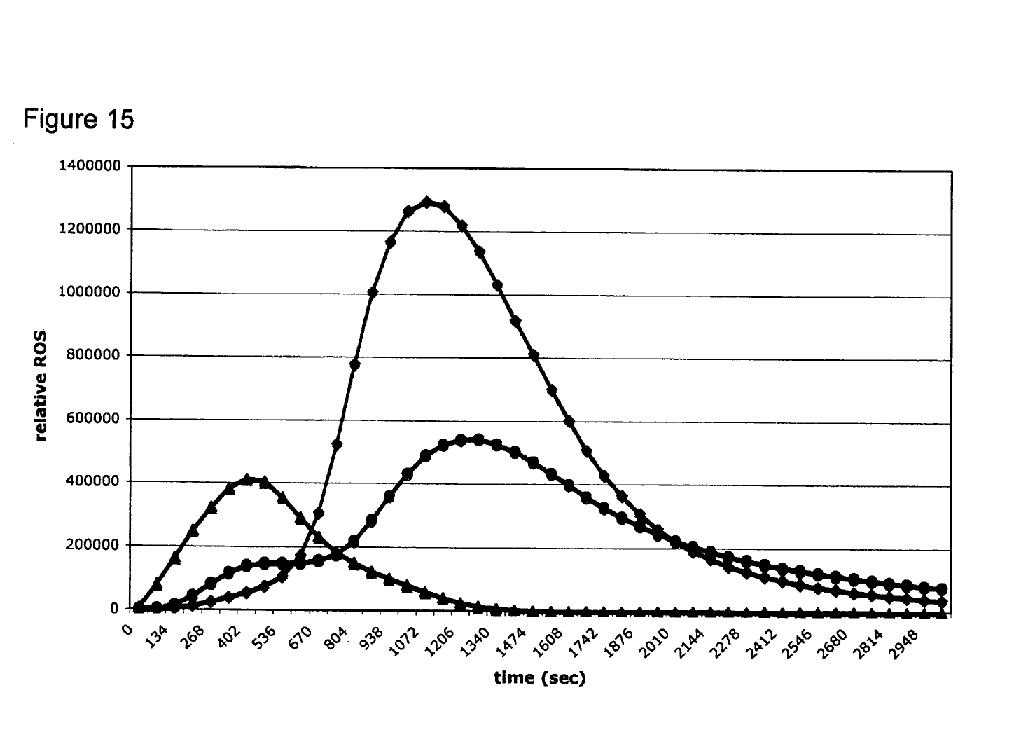

The NADPH activators compound 1 (phytol), compound 2 (farnesol) and compound 9 (isophytol) give strong ROS production from the HL60 cells. Extra-cellular ROS production was measured with an isoluminol assay on HL-60 cells after in vitro stimulation with compound 1 (phytol) (♦), compound 2 (farnesol) (▴) or compound 9 (isophytol) (●) (see FIG. 15).

### Example 17

Arthritis Treatment by Oral Gavage With Compound 1 (Phytol) at 1.6 or 2.3 g/kg/day

Pristane induced arthritis (PIA) was induced in the rats by a subcutaneous (s.c.) injection at the base of the tail with 200 μl of a 50% mixture of pristane/hexadecane. Treatments with compound 1 (phytol) at 1.6 or 2.3 g/kg/day administered to DA.Ncf1^{DA} rats by oral gavage given by dosing every second day with compound 1 (phytol) 1.6 mg/kg/every second day (♦), compound 1 (phytol) 3.2 mg/kg/every second day (▴) or untreated control (◯).

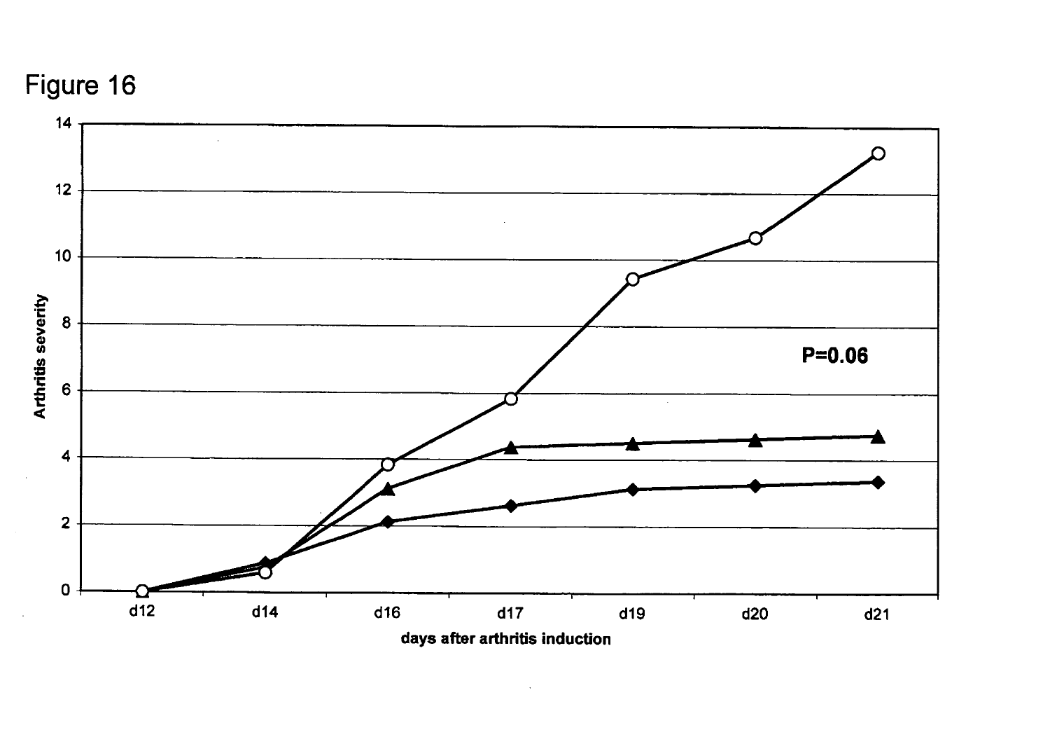

Both doses of compound 1 (phytol) given by oral gavage have potent arthritis ameliorating effect. No adverse effect of the high doses given was observed, either by macroscopically observations or at postmortem dissection examination (see FIG. 16).

### Example 18

Arthritis Treatment With Oral Administration of Compound 1 (Phytol) in Comparison With Fenofibrate

Fenofibrate (propan-2-yl 2-[4-(4-chlorobenzoyl)phenoxy]-2-methyl-propanoate; Sigma-Aldrich) is known to be an antilipemic agent. It has been suggested that fenofibrate suppresses the development of arthritis by inhibition of NF-kappa B signaling. See, e.g., Okamoto et al. CLIN EXP RHEUMATOL 23 (3): 323-330 May-June 2005.

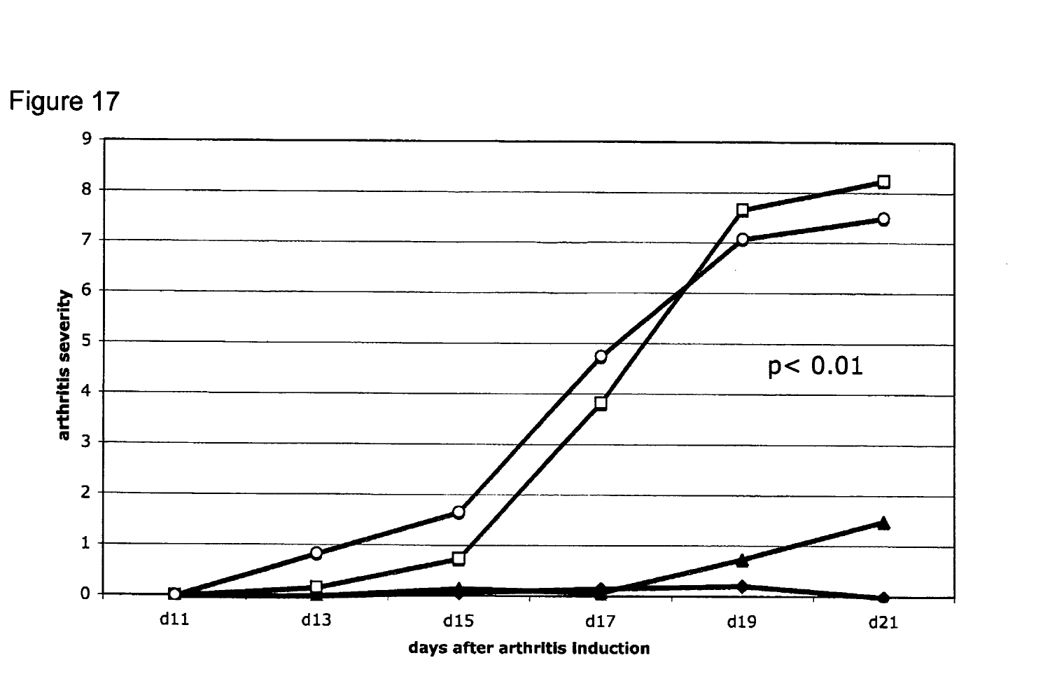

Pristane induced arthritis (PIA) was induced in the rats by a subcutaneous (s.c.) injection at the base of the tail with 200 μl of a 50% mixture of pristane/hexadecane. Treatments of DA.Ncf1^{DA} rats with compound 1 (phytol) or compound 9 (isophytol) mixed in the food for continuous uptake compared with fenofibrate, (compound 1 (phytol) 500 mg/kg/day (♦), compound 9 (isophytol) 500 mg/kg/day (▴), or fenofibrate 100 mg/kg/day (□). Untreated control (◯).) (see FIG. 17).

Both treatments by oral administration with compound 1 (phytol) or compound 9 (isophytol) reduced the severity of arthritis in the animals. Treatment with fenofibrate had no effect on the severity of arthritis.

### Example 19

Treatment of Experimental Allergic Encephalomyelitis (EAE) In Vivo

At day 0, all rats were treated (immunized) with 200 μL SCH (DA.Ncf1^{DA} spinal cord homogenate, to induce EAE) i.d. at the base of the tail. The rats were then scored according to the following scale for 40 days:
0=Normal
1=Tail weakness
2=Tail paralysis
3=Tail paralysis and mild waddle
4=Tail paralysis and severe waddle
5=Tail paralysis and paralysis of one limb
6=Tail paralysis and paralysis of a pair of limbs
7=Tetra-paresis
8=Pre-morbid or dead.

One group of DA.Ncf1^{DA} rats (N=6/group) was injected at the base of the tail with 200 μL of compound 1 (phytol) at day 0.

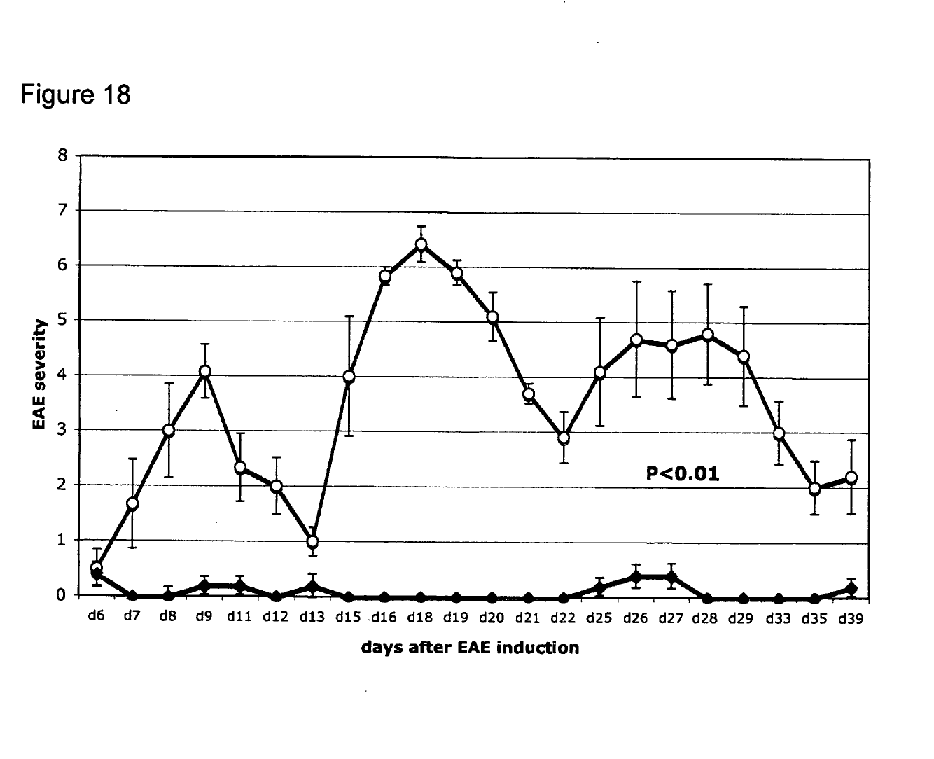

EAE began to appear around day 6. The results show a significant difference in severity of inflammatory and paralyzing symptoms of EAE (p<0.01) between the rats treated with compound 1 (phytol) (♦) or untreated control (◯), (see FIG. 18).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating an autoimmune condition in a mammal in need thereof, said method comprising administering to said mammal a compound, or a pharmaceutically acceptable salt thereof, that enhances NADPH oxidase activity, wherein said compound comprises one or more isoprenoid units.

2. The method of claim 1, wherein said autoimmune condition is arthritis.

3. The method of claim 1, wherein said autoimmune condition is multiple sclerosis.

4. The method of claim 1, wherein said compound comprises one or more unsaturated isoprenoid units.

5. The method of claim 1, wherein said compound comprises one or more saturated isoprenoid units.

6. The method of claim 4 or 5, wherein at least one of said unsaturated isoprenoid units or saturated isoprenoid units is derivatized with a functional moiety.

7. The method of claim 1, wherein the compound has the following general formula: 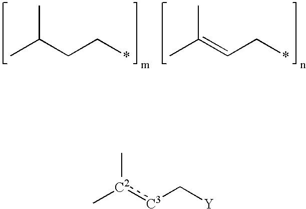
wherein:
m+n=an integer from 0-9; [symbol]
represents a single or double bond between C² and C³; and
Y is —OR′ or —O—C(═O)R′, wherein R′ is H, alkyl, alkenyl, alkynyl, cycloalkyl, heterocyclyl, aryl, or heteroaryl, each of which is optionally substituted.

8. The method of claim 7, wherein Y is OR′.

9. The method of claim 8, wherein R′ is hydrogen.

10. The method of claim 7, wherein Y is —O—C(═O)R′.

11. The method of claim 10, wherein R′ is C₁-C₆ alkyl.

12. The method of claim 11, wherein R′ is CH₃.

13. The method of claim 7, wherein m+n=an integer from 0-5.

14. The method of claim 7, wherein one of m and n is 0, and the other is an integer from 1-5.

15. The method of claim 7, wherein m+n=3.

16. The method of claim 15, wherein one of m and n is 0.

17. The method of claim 16, wherein n is 0.

18. The method of claim 7, wherein m+n=2.

19. The method of claim 7, wherein [symbol] represents a double bond between C² and C³.

20. The method of claim 7, wherein the compound is selected from the group consisting of:
(3,7,11,15-Tetramethyl-2-hexadecen-1-ol);
(3,7,11-Trimethyl-2,6,10-dodecatrien-1-ol);
(3,7,11,15-Tetramethyl-hexada-2,6,10,14-tetraen-1-ol);
(Acetic acid 3,7,11-trimethyl-dodeca-2,6,10-trienyl ester);
(Acetic acid 3,7,11,15-tetra-metyl-hexadec-2-enyl-ester);
(3,7,11,15-Tetramethyl-hexadecan-1-ol); and
(Acetic acid 3,7,11,15-tetramethyl-hexadecyl ester).

21. The method of claim 7, wherein the compound is (3,7,11,15-Tetramethyl-2-hexadecen-1-ol).

22. The method of claim 1, wherein the compound has the general formula: 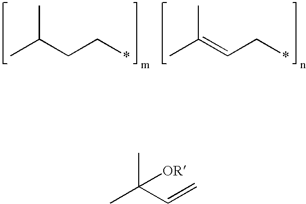
wherein n+m=an integer from 0 to 9; and R′ is H, alkyl, alkenyl, alkynyl, cycloalkyl, heterocyclyl, aryl, or heteroaryl, each of which is optionally substituted.

23. The method of claim 22, wherein m+n=3.

24. The method of claim 23, wherein n is 0.

25. The method of claim 22, wherein R′ is hydrogen.

26. The method of claim 22, wherein the compound is 3,7,11,15-tetramethyl-1-hexadecen-3-ol.

27. The method of claim 1, wherein the compound has the general formula: 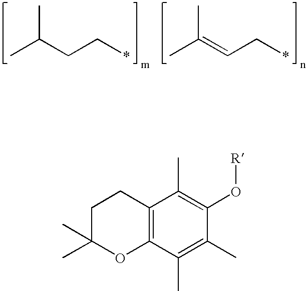
wherein n+m=an integer from 0 to 9; and R′ is H, alkyl, alkenyl, alkynyl, cycloalkyl, heterocyclyl, aryl, or heteroaryl, each of which is optionally substituted.

28. The method of claim 27, wherein m+n=3.

29. The method of claim 28, wherein n is 0.

30. The method of claim 27, wherein R′ is C₁-C₃ alkyl substituted with from 1-3 halogens.

31. The method of claim 27, wherein the compound is 6-Difluoromethoxy-2,5,7,8-tetramethyl-2-(4,8,12-trimethyl-tridecyl)-chroman.

32. The method of claim 1, wherein said composition is administered intra-dermally, intra-peritoneally, orally, or intra-nasally.
